(19) United States
(12) Patent Application Publication
(10) Pub. No.: US 2021/0171929 A1
(43) Pub. Date: Jun. 10, 2021

(54) SINGLE BASE EDITING TOOLS WITH PRECISE ACCURACY

(71) Applicant: William Marsh Rice University, Houston, TX (US)

(72) Inventors: Xue GAO, Houston, TX (US); Sangsin LEE, Houston, TX (US); Ning DING, Houston, TX (US)

(73) Assignee: William Marsh Rice University, Houston, TX (US)

(21) Appl. No.: 17/104,562

(22) Filed: Nov. 25, 2020

Related U.S. Application Data
(60) Provisional application No. 62/940,017, filed on Nov. 25, 2019.

(51) Int. Cl. C12N 9/78; C12N 9/22; C12N 15/11; C12N 15/90

(52) U.S. Cl. CPC C12N 9/78; C12Y 305/04001; C12N 9/22; C12N 15/11; A61K 38/00; C12N 2800/80; C12N 2310/20; C07K 2319/00; C12N 15/907

(57) ABSTRACT

Provided herein are systems, reagents, methods, and kits that are useful for the targeted editing of nucleic acids, including editing a single site within the genome of a cell or subject, e.g., within the human genome. In some embodiments, fusion proteins of Cas9 and cytosine deaminase domains, are provided. In some embodiments, methods for targeted nucleic acid editing are provided.

### REFERENCE TO RELATED APPLICATIONS

The present application claims the priority benefit of U.S. provisional application No. 62/940,017, filed Nov. 25, 2019, the entire contents of which is incorporated herein by reference.

### REFERENCE TO A SEQUENCE LISTING

The instant application contains a Sequence Listing, which has been submitted in ASCII format via EFS-Web and is hereby incorporated by reference in its entirety. Said ASCII copy, created on Nov. 12, 2020, is named RICEP0071US_ST25.txt and is 855.3 kilobytes in size.

### BACKGROUND

### 1. Field

The present invention relates generally to the fields of molecular biology and medicine. More particularly, it concerns fusion proteins for the targeted conversion of cytosine nucleotides to thymine nucleotides and methods of using said fusion proteins to edit nucleic acids and treat disease.

### 2. Description of Related Art

Nucleobase editing technology enables conversion of cytosine (C) to thymine (T) on a specific DNA target (see, e.g., U.S. Pat. No. 10,167,457). The base editors used in this technology comprise (1) an RNA-programmable DNA-targeting protein (i.e., Cas9), (2) a guide RNA that directs the DNA-targeting protein to the intended target site, and (3) a cytosine deaminase enzyme that converts the target C to uridine (U), which is then converted to T by the endogenous cellular repair pathway. All existing base editors have a defined activity window size, typically 5-nucleotides, within the DNA target region, which defines the range of Cs the base editor can cover to convert to Ts. Any Cs positioned within this window could be converted to T, including any non-target Cs, thus leading to unwanted C-to-T byproducts. Researchers have tried decreasing the window size to reduce such byproducts, but this strategy greatly limits the targeting scope of base editors because it allows only a particularly preferable position of C within the window to be converted, leaving Cs located on the other non-preferable positions, which might be the actual main target, to be non-targetable.

### SUMMARY

In one embodiment, provided herein are polypeptides comprising a variant of a native cytosine deaminase (CD) domain, wherein the variant CD domain comprises a sequence at least 90%, at least 95%, at least 98%, or 100% identical to amino acids 198-384 of SEQ ID NO: 48, and comprises P200A, N236A, P247K, Q318K, Q322K substitutions relative to SEQ ID NO: 48. In some aspects, the polypeptides further comprise a Y315F or a N244G substitution relative to SEQ ID NO: 48. In some aspects, the polypeptides further comprise H248N, K249L, H250L, G251C, F252G, L253F, and E254Y substitutions relative to SEQ ID NO: 48. In some aspects, the polypeptides further comprise a Y315F substitution relative to SEQ ID NO: 48. In some aspects, the polypeptides further comprise a D316H substitution relative to SEQ ID NO: 48, a reversion back to proline at position 247 of SEQ ID NO: 48, or a reverse back to glutamine at position 318 of SEQ ID NO: 48. In some aspects, the polypeptides further comprise a E217K, T311A, or R210L substitution relative to SEQ ID NO: 48. In some aspects, the polypeptides further comprise T311A and R320L substitutions relative to SEQ ID NO: 48. In some aspects, the polypeptides further comprise a E217K substitution relative to SEQ ID NO: 48. In some aspects, the polypeptides further comprise a N244Q, S286A, D316E, R313A, Y315F, S286A, N244G, W285Y, R320E, D316R, D317R, Y315L, or Y315W substitution relative to SEQ ID NO: 48.

In some aspects, the polypeptides further comprise L234K, F310K, C243A, C321A, and C356A substitutions relative to SEQ ID NO: 48. In some aspects, the polypeptides further comprise a Y315F substitution relative to SEQ ID NO: 48. In some aspects, the polypeptides further comprise a W285Y, W285F, R320A, R320E, or R326E substitution relative to SEQ ID NO: 48. In some aspects, the polypeptides further comprise an R320E substitutions relative to SEQ ID NO: 48.

In some aspects, the CD domain is a chimpanzee, gorilla, monkey, cow, dog, rat, mouse, or human APOBEC3G deaminase domain. In some aspects, the polypeptides further comprise a Cas9 domain that has nickase activity. In some aspects, the Cas9 domain is Streptococcus pyogenes Cas9 (SpCas9), Staphylococcus aureus Cas9 (SaCas9), Cas9-VQR, Cas9-VRER, SaCas9, StCas9, NmCas9, CjCas9, CasX, CasY, Cpf1, C2c1, C2c2, or C2c3. In some aspects, the Cas9 domain is a Streptococcus pyogenes Cas9 (SpCas9) having a D 10A substitution. In some aspects, the Cas9 domain is positioned at the C-terminus of the CD domain. In some aspects, the Cas9 domain comprises a sequence at least 90%, at least 95%, at least 98%, or 100% identical to amino acids 435-1801 of SEQ ID NO: 3.

In some aspects, a linker is positioned between the CD domain and the Cas9 domain. In some aspects, the linker is a 32 amino acid GSSG linker. In some aspects, the linker comprises a sequence of amino acids 403-434 of SEQ ID NO: 3. In some aspects, the linker is a 3 amino acid PAP linker. In some aspects, the linker is a 7 amino acid repetitive proline-alanine (PA) linker. In some aspects, the linker is a 9 amino acid repetitive proline-alanine (PA) linker. In some aspects, the linker is a 15 amino acid repetitive proline-alanine (PA) linker.

In some aspects, the polypeptides further comprise a uracil glycosylase inhibitor (UGI) domain. In some aspects, the UGI domain is positioned at the C-terminus of the Cas9 domain. In some aspects, the UGI domain comprises a sequence at least 90%, at least 95%, at least 98%, or 100% identical to amino acids 1812-1987 of SEQ ID NO: 3. In some aspects, a linker is positioned between the Cas9 domain and the UGI domain. In some aspects, the linker comprises a sequence of amino acids 1802-1811 of SEQ ID NO: 3.

In some aspects, the polypeptides further comprise a nuclear localization sequence. In some aspects, the nuclear localization sequence is a bipartite nuclear localization sequence.

In one embodiment, provided herein are nucleic acids comprising a nucleotide sequence encoding the polypeptide of any one of the present embodiments. In some aspects, the nucleic acid is codon optimized for expression in bacteria, fungus, insects, or mammals. In some aspects, the nucleic acid is codon optimized for expression in a human cell.

In one embodiment, provided herein are expression vectors comprising the nucleic acid of any one of the present embodiments. In some aspects, the nucleotide sequence encoding the polypeptide is operably linked to a first expression control element. In some aspects, the expression vectors further comprise a nucleotide sequence encoding a guide RNA (gRNA). In some aspects, the nucleotide sequence encoding the gRNA is operably linked to a second expression control element.

In one embodiment, provided herein are host cells comprising the nucleic acid of any one of the present embodiments. In some aspects, the host cell is a bacterial cell, a fungal cell, an insect cell, or a mammalian cell. In some aspects, the host cell is a human cell.

In one embodiment, provided herein are pharmaceutical formulations comprising the nucleic acid of any one of the present embodiments in a pharmaceutically acceptable carrier.

In one embodiment, provided herein are viral vectors comprising the nucleic acid of any one of the present embodiments. In some aspects, the viral vector is an adeno-associated viral (AAV) vector, a lentiviral vector, or a retroviral vector. In some aspects, the viral vector further comprises a nucleotide sequence encoding a guide RNA (gRNA). In some aspects, the nucleotide sequence encoding the gRNA is operably linked to a second expression control element. In some aspects, the first and/or second expression control element comprises a promoter. In some aspects, the first and/or second expression control element comprises an enhancer element. In some aspects, the viral vectors further comprise one or more of an intron, a filler polynucleotide sequence, and/or poly A signal.

In one embodiment, provided herein are compositions comprising the nucleic acid of any one of the present embodiments and a nucleic acid encoding a guide RNA. In some aspects, the gRNA comprises a fusion of a CRISPR-RNA (crRNA) and a trans-activating CRISPR RNA (tracrRNA). In some aspects, the gRNA comprises a crRNA and a tracrRNA. In some aspects, the gRNA is a 5′ extended gRNA. In some aspects, the nucleic acid encoding the guide RNA is codon optimized for expression in bacteria, fungus, insects, or mammals. In some aspects, the nucleic acid encoding the gRNA is codon optimized for expression in a human cell. In some aspects, the nucleotide sequence encoding the polypeptide is operably linked to a first expression control element. In some aspects, the nucleotide sequence encoding the gRNA is operably linked to a second expression control element. In some aspects, the first and/or second expression control element comprises a promoter. In some aspects, the first and/or second expression control element comprises an enhancer element.

In one embodiment, provided herein are compositions comprising the polypeptide of any one of the present embodiments and a guide RNA bound to the Cas9 domain of the polypeptide. In some aspects, the gRNA comprises a fusion of a CRISPR-RNA (crRNA) and a trans-activating CRISPR RNA (tracrRNA). In some aspects, the gRNA comprises a crRNA and a tracrRNA. In some aspects, the gRNA is a 5′ extended gRNA.

In one embodiment, provided herein are methods for targeted modification of a selected DNA sequence, the method comprising contacting the DNA sequence with a polypeptide of any one of the present embodiments and a nucleic acid comprising a guide RNA (gRNA) sequence targeted to the selected DNA sequence, where the gRNA complexed with the polypeptide and directs the polypeptide to the selected DNA sequence. In some aspects, targeted modification is the deamination of a deoxycytidine within the selected DNA sequence. In some aspects, the selected sequence comprises a CC motif. In some aspects, the targeted modification is the deamination of the second deoxycytidine within the CC motif. In some aspects, the selected sequence comprises a DCYD motif, wherein D represent A, G, or T, and wherein Y denotes the T>C mutation. In some aspects, the selected sequence comprises a DCCYD motif, wherein D represent A, G, or T, and wherein Y denotes the T>C mutation. In some aspects, the selected sequence comprises a CCCA motif, wherein the targeted modification is the deamination of the third deoxycytidine within the motif.

In some aspects, the introducing comprises introducing into the cell a nucleic acid encoding the polypeptide, and wherein the method further comprises maintaining the cell under conditions in which the nucleic acid encoding the polypeptide is expressed. In some aspects, the contacting is in vitro. In some aspects, the contacting is in vivo in a subject identified as having a clinical condition. In some aspects, the selected DNA sequence is associated with the clinical condition, and wherein deamination of the cytidine results in a sequence that is not associated with the clinical condition. In some aspects, the clinical condition is hereditary pyropoikilocytosis, cystic fibrosis, or holocarboxylase synthetase deficiency. In some aspects, the deamination corrects a point mutation in the selected DNA sequence associated with the clinical condition. In some aspects, the selected DNA sequence comprises a T to C point mutation associated with the clinical condition, and wherein the deamination of the mutant C base results in a sequence that is not associated with a disease or disorder. In some aspects, the selected DNA sequence encodes a protein, and wherein deamination of the nucleotide base results in a correction of a T to C point mutation to restore the wild-type sequence of the encoded protein.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1. Graphical representation of the results of testing the C-to-T editing abilities of the engineered A3G base editors on the GCCCA motif in the HEK3 site.

FIG. 2. Graphical representation of the results of testing the C-to-T editing abilities of the engineered A3G base editors on the ACCA motif in the HEK4 #a2 site.

FIG. 3. Graphical representation of the results of testing the C-to-T editing abilities of the engineered A3G base editors on the TCCA motif in the EMX1 #2 site.

FIG. 4. Graphical representation of the results of testing the C-to-T editing abilities of the engineered A3G base editors on the TCCA motif in the RNF2 #2 site.

FIGS. 5A-5C. Graphical representation of the results of testing the C-to-T editing abilities of the engineered A3G base editors on the GCCT motif in the BSC1L #1 site, the ACCT motif in the PPP 1R12C #a2 site, and the TCCT motif in the EMX1 #a3 site.

FIG. 6. Graphical representation of the results of testing the C-to-T editing abilities of the engineered A3G base editors on the ACCA motif in the AMX1 #a10 site.

FIG. 7. Graphical representation of the results of testing the C-to-T editing abilities of the engineered A3G base editors on the GCCG motif in the FANCF #a3 site.

FIG. 8. Graphical representation of the results of testing the C-to-T editing abilities of the engineered A3G base editors on the GCCG motif in the EMX1 #a18 site.

FIG. 9. Graphical representation of the results of testing the C-to-T editing abilities of the engineered A3G base editors on the GCCG and ACCA motifs in the FANCF #2 site.

FIG. 10. Graphical representation of the results of testing the C-to-T editing abilities of the engineered A3G base editors on the ACCG motif in the HEK4 #a1 site.

FIGS. 11-13. Graphical representations of the results of testing the C-to-T editing abilities of the engineered A3G base editors on the TCCG motif in the EMX1 site.

FIG. 14. Graphical representation of the results of testing the C-to-T editing abilities of the engineered A3G base editors on the ACACA motif in the HEK2 site.

FIG. 15. Graphical representation of the results of testing the C-to-T editing abilities of the engineered A3G base editors on the TCGCCG motif in the MSSK1-M-c site.

FIG. 16. Graphical representation of the results of testing the C-to-T editing abilities of the engineered A3G base editors on the TCCCT motif in the FANCF site.

FIG. 17. Graphical representation of the results of screening the base editing efficiency and specificity of engineered A3G-BE variants on the EMX1 and FANCF #a3 sites in HEK293T cells.

FIG. 18. Representation of the HTS analysis performed to quantify modified alleles for disease modeling and correction after treatment with BE4max, A3G-BE4.4, A3G-BE5.13, and A3G-BE5.14.

FIG. 19. A3G-BE5.13 with altered linkers.

FIG. 20. Graphical representation of the results of screening the base editing efficiency and specificity of engineered A3G-5.13 variants on the VEGF #2 and EMX1 PolyC #1 sites in HEK293T cells.

### DETAILED DESCRIPTION

The present process for base editing involves utilizing a different cytosine deaminase homolog, called APOBEC3G (or A3G), that has a unique native property of recognizing the ‘CC’ dinucleotide sequence motif on DNA. In the sequence context of two consecutive ‘CC,’ A3G has a preference to deaminate the latter C, thereby providing for the conversion of a single C-to-T when an immediate bystander C is present. To allow for precise discrimination between two or possibly more consecutive Cs for the induction of clean single C-to-T conversions without the generation of unwanted byproduct(s), A3G has been engineered herein to enhance its basal activity level and recover its native base editing preference for the ‘CC’ sequence motif. The overall enzymatic activity of the A3G engineered variants was increased by increasing its expression level as well as by introducing selected mutations in the A3G deaminase portion of the enzyme. In addition, residues that can be engineered to modulate the enzyme's sensitivity for ‘CC’ selectivity were identified. The engineered A3G base editors provided herein (see Table 1) allow for a clean C-to-T conversion in the presence of multiple consecutive Cs regardless of the size of the activity window.

APOBEC3G orthologs originating from chimpanzee, gorilla, monkey, cow, dog, rat, mouse, or other mammals may replace the human APOBEC3G deaminase portion of any base editor while harboring one or more corresponding mutations to those provided herein. Base editors using different cytidine deaminase enzymes that also perform single C-to-T conversions in the context of consecutive Cs is also contemplated.

The present engineered A3G base editors, in principle, can discriminate between two consecutive Cs (C₋₁ and C₀ in the DNA context of ‘C₋₁C₀’) when they are positioned within the base editing activity window. It can be used as a CRISPR-based genome editing tool to cleanly install a single C-to-T mutation on the target C₀ while leaving C₋₁ unmutated, making C₋₁T₀ the most likely outcome using the present engineered A3G base editors. If other base editors lacking the ‘CC’ preferential motif are used, T₋₁T₀, C₋₁T₀, and T₋₁C₀ would all have approximately the equal chance to be the outcomes. In case of more than two Cs, for example three consecutive ‘C₋₂C₋₁C₀’, it is important to note that the first and the second Cs (C₋₂C₋₁) constitute the ‘CC’ dinucleotide motif, and therefore C₋₁ would be converted to T while C₋₂ would not. The same principle applies to the second and third Cs (C₋₁C₀) in this context: C₀ would be converted to T while C₋₁ would not. As a result, in the context of three consecutive Cs, any combinations of C-to-T conversions on the positions of C₋₁ and C₀ could be possible, making ‘C₋₂T₋₁T₀,’ ‘C₋₂C₋₁T₀,’ ‘C₋₂T₋₁C₀,’ to be the highest possible outcomes. If other base editors lacking the ‘CC’ preferential motif are used, C₋₂ could also be converted to T, making ‘C₋₂C₋₁T₀,’ ‘C₋₂T₋₁C₀,’ ‘T₋₂C₋₁C₀,’ ‘C₋₂T₋₁T₀,’ ‘T₋₂C₋₁T₀,’ ‘T₋₂T₋₁C₀,’ and ‘T₋₂T₋₁T₀’ to be the all possible outcomes. As a general principle, the very first C in the sequence context of several consecutive Cs would not be converted to T when using the present engineered A3G base editors, while other base editors would also convert the very first C to T because they lack the ‘CC’ preferential motif. In addition, certain of the present engineered A3G base editors retain high editing efficiency at C₀ while having diminished activity at other positions falling within the activity window.

The C-to-T conversion rate also depends on the overall sequence context as well as the chromatin structure of the target DNA. For example, studies suggest the ideal preferential motif of A3G is ‘CCCA.’ The conversion rate of C-to-T could be much higher in this full ideal motif, while the result in the context of ‘CC’ dinucleotide motif may vary across different genomic sites depending on the nearby sequence context. The accessibility of the target DNA depending on its endogenous structure could also affect the conversion rate. For example, heterochromatin refers to tightly packed DNA structure. This could influence the accessibility of base editors to the target DNA and therefore influencing the C-to-T conversion rate.

The correction of genetic mutations associated with diseases requires high precision and accuracy. For example, the engineered A3G base editors can cleanly correct pathogenic T>C disease mutations when such point mutations are positioned within the context of a ‘DCYD’ or ‘DCCYD’ sequence motif, in which Y denotes the T>C mutation position and D denotes any nucleotide other than C (i.e., A, G, or T). For such therapeutic applications, the engineered base editors provided herein can be packaged into delivery vehicles such as lipofectamine, AAV vector, lentivirus, and others to treat cells for C-to-T conversions that are within the engineered enzyme's selectivity specifications.

TABLE 1
A3G Base Editor Variants

| | A3G | | | | |
|---|---|---|---|---|---|
| A3G-BE | codon- | | | | SEQ ID |
| Versions | optimized | A3G Domains | Backbone | A3G Specification/Mutation** | NO*** |
| hA3G-BE3 | x | NTD + CTD | BE3 | — | 1 |
| 1.1 | x | NTD + CTD | BE4 | — | 2 |
| 1.2 | x | NTD + CTD | BE4MAX | — | 3 |
| 2.1 | ∘ | NTD + CTD | BE4MAX | — | 4 |
| 2.2 | ∘ | NTD + CTD | BE4MAX | N244G | 5 |
| 2.3 | ∘ | NTD + CTD | BE4MAX | Y315F | 6 |
| 3.1 | ∘ | NTD + CTD | BE4MAX | (P200A + N236A + P247K + | 7 |
| | | | | Q318K + Q322K) | |
| 3.2 | ∘ | NTD + CTD | BE4MAX | (P200A + N236A + P247K + | 8 |
| | | | | Q318K + Q322K) + (N244G) | |
| 3.3 | ∘ | NTD + CTD | BE4MAX | (P200A + N236A + P247K + | 9 |
| | | | | Q318K + Q322K) + (Y315F) | |
| 4.1 | ∘ | NTD + CTD | BE4MAX | (P200A + N236A + P247K + | 10 |
| | | | | Q318K + Q322K) + (A3A loop3) | |
| 4.2 | ∘ | NTD + CTD | BE4MAX | (P200A + N236A + P247K + | 11 |
| | | | | Q318K + Q322K) + (L234K + | |
| | | | | C243A + F310K + C321A + | |
| | | | | C356A) | |
| 4.3 | ∘ | NTD + CTD | BE4MAX | (P200A + N236A + P247K + | 12 |
| | | | | Q318K + Q322K) + (A3A loop3) + | |
| | | | | (L234K + C243A + F310K + | |
| | | | | C321A + C356A) | |
| 4.4 | ∘ | 198-384 (CTD) | BE4MAX | — | 13 |
| 4.5 | ∘ | 198-384 (CTD) | BE4MAX | D316R/D317R | 14 |
| 5.1 | ∘ | 198-384 (CTD) | BE4MAX | (P200A + N236A + P247K + | 15 |
| | | | | Q318K + Q322K) | |
| 5.2 | ∘ | 175-384 | BE4MAX | (P200A + N236A + P247K + | 16 |
| | | (alpha6 NTD + CTD) | | Q318K + Q322K) | |
| 5.3 | ∘ | 198-384 (CTD) | BE4MAX | (P200A + N236A + P247K + | 17 |
| | | | | Q318K + Q322K) + (A3A loop3)* | |
| 5.4 | ∘ | 198-384 (CTD) | BE4MAX | (P200A + N236A + P247K + | 18 |
| | | | | Q318K + Q322K) + (L234K + | |
| | | | | F310K + C243A + C321A + | |
| | | | | C356A) | |
| 5.5 | ∘ | 198-384 (CTD) | BE4MAX | (P200A + N236A + P247K + | 19 |
| | | | | Q318K + Q322K) + (A3A loop3) + | |
| | | | | (L234K + F310K + C243A + | |
| | | | | C321A + C356A) | |
| 5.6 | ∘ | 198-384 (CTD) | BE4MAX | (P200A + N236A + P247K + | 20 |
| | | | | Q318K + Q322K) + (A3A loop3) + | |
| | | | | Q322A | |
| 5.7 | ∘ | 198-384 (CTD) | BE4MAX | (P200A + N236A + P247K + | 21 |
| | | | | Q318K + Q322K) + (A3A loop3) + | |
| | | | | R320L | |
| 5.8 | ∘ | 198-384 (CTD) | BE4MAX | (P200A + N236A + P247K + | 22 |
| | | | | Q318K + Q322K) + (A3A loop3) + | |
| | | | | T311A | |
| 5.9 | ∘ | 198-384 (CTD) | BE4MAX | (P200A + N236A + P247K + | 23 |
| | | | | Q318K + Q322K) + (A3A loop3) + | |
| | | | | E217K | |
| 5.10 | ∘ | 198-384 (CTD) | BE4MAX | (P200A + N236A + P247K + | 24 |
| | | | | Q318K + Q322K) + (A3A loop3) + | |
| | | | | (T311A + R320L) | |
| 5.11 | ∘ | 198-384 (CTD) | BE4MAX | (P200A + N236A + P247K + | 25 |
| | | | | Q318K + Q322K) + (A3A loop3) + | |
| | | | | (T311A + R320L + E217K) | |
| 5.12 | ∘ | 198-384 (CTD) | BE4MAX | A3G 5.1 + Y315F | 26 |
| 5.13 | ∘ | 198-384 (CTD) | BE4MAX | A3G 5.3 + Y315F | 27 |
| 5.14 | ∘ | 198-384 (CTD) | BE4MAX | A3G 5.4 + Y315F | 28 |
| 5.20 | ∘ | 198-384 (CTD) | BE4MAX | A3G 5.13 with a PAP 3aa spacer | 49 |
| 5.21 | ∘ | 198-384 (CTD) | BE4MAX | A3G 5.20 + W285Y | 50 |
| 5.22 | ∘ | 198-384 (CTD) | BE4MAX | A3G 5.20 + W285F | 51 |
| 5.26 | ∘ | 198-384 (CTD) | BE4MAX | A3G 5.20 + R320A | 52 |
| 5.28 | ∘ | 198-384 (CTD) | BE4MAX | A3G 5.20 + R320E | 53 |
| 5.29 | ∘ | 198-384 (CTD) | BE4MAX | A3G 5.20 + R326E | 54 |
| 6.11 | ∘ | 198-384 (CTD) | BE4MAX | A3G 5.10 + Y315F | 29 |
| 6.12 | ∘ | 198-384 (CTD) | BE4MAX | A3G 5.10 + N244G | 30 |
| 6.13 | ∘ | 198-384 (CTD) | BE4MAX | A3G 5.10 + D316E | 31 |
| 6.14 | ∘ | 198-384 (CTD) | BE4MAX | A3G 5.10 + D316R + D317R | 32 |
| 6.15 | ∘ | 198-384 (CTD) | BE4MAX | A3G 5.10 + W285Y + R320E | 33 |
| 6.16 | ∘ | 198-384 (CTD) | BE4MAX | A3G 5.10 + Y315W | 34 |
| 6.17 | ∘ | 198-384 (CTD) | BE4MAX | A3G 5.10 + Y315L | 35 |
| 6.18 | ∘ | 198-384 (CTD) | BE4MAX | A3G 6.11 + N244Q | 36 |
| 6.19 | ∘ | 198-384 (CTD) | BE4MAX | A3G 6.11 + S286A | 37 |
| 6.20 | ∘ | 198-384 (CTD) | BE4MAX | A3G 6.11 + R313A | 38 |
| 6.21 | ∘ | 198-384 (CTD) | BE4MAX | A3G 6.11 + original A3G loop3 | 39 |
| 6.22 | ∘ | 198-384 (CTD) | BE4MAX | A3G 5.10 + R313A | 40 |
| 6.23 | ∘ | 198-384 (CTD) | BE4MAX | A3G 5.10 + N244Q | 41 |
| 6.24 | ∘ | 198-384 (CTD) | BE4MAX | A3G 5.10 + S286A | 42 |
| 7.1 | ∘ | 198-384 (CTD) | BE4MAX | A3G 5.13 + D316H | 43 |
| 7.5 | ∘ | 198-384 (CTD) | BE4MAX | A3G 5.13 + P247K-->P | 44 |
| 7.6 | ∘ | 198-384 (CTD) | BE4MAX | A3G 5.13 + Q318K-->Q | 45 |
| 8.1 | ∘ | 198-384 (CTD) | BE4MAX | A3G 5.13 + HypaCas9 | 46 |
| | | | | (Cas9: N692A + M694A + | |
| | | | | Q695A + H698A) | |

*A3A loop3 mutation includes P247K, H248N, K249L, H250L, G251C, F252G, L253F, E254Y. Note that P247K is already included in the first set of five mutations.
**Note that the positions of the amino acid substitutions are all relative to positions in SEQ ID NO: 48.
***For SEQ ID NO: 1, positions 2-197 represent the NTD of A3G, positions 198-400 represent the CTD of A3G, positions 401-1865 represent cas9 nickase, positions 1866-1771 represent a linker sequence, and positions 1772-1854 represent UGI; for each of SEQ ID NOs: 3-12, positions 2-19 represent one part of a bipartite nuclear localization sequence, positions 20-15 represent the NTD of A3G, positions 216-402 represent the CTD of A3G, positions 402-434 represent a linker sequence, positions 435-1801 represent cas9 nickase, positions 1802-1811 represent a linker sequence, positions 1812-1894 represent a first occurrence of UGI, positions 1895-1904 represent a linker sequence, 1905-1987 represent a second occurrence of UGI, positions 1988-1991 represent a linker sequence, and positions 1992-2008 represent the second part of a bipartite nuclear localization sequence; for each of SEQ ID NOs: 13-28, positions 2-19 represent one part of a bipartite nuclear localization sequence, positions 20-206 represent the CTD of A3G, positions 207-238 represent a linker sequence, positions 239-1605 represent cas9 nickase, positions 1606-1615 represent a linker sequence, positions 1616-1698 represent a first occurrence of UGI, positions 1699-1708 represent a linker sequence, 1709-1791 represent a second occurrence of UGI, positions 1792-1795 represent a linker sequence, and positions 1796-1812 represent the second part of a bipartite nuclear localization sequence; for SEQ ID NO: 16, positions 2-19 represent one part of a bipartite nuclear localization sequence, positions 20-229 represent the alpha6 NTD + CTD of A3G, positions 230-261 represent a linker sequence, positions 262-1628 represent cas9 nickase, positions 1629-1638 represent a linker sequence, positions 1639-1721 represent a first occurrence of UGI, positions 1722-1731 represent a linker sequence, 1732-1814 represent a second occurrence of UGI, positions 1815-1818 represent a linker sequence, and positions 1819-1835 represent the second part of a bipartite nuclear localization sequence; for SEQ ID NOs: 49-54, positions 2-19 represent one part of a bipartite nuclear localization sequence, positions 20-206 represent the CTD of A3G, positions 207-209 represent a linker sequence, positions 210-1576 represent cas9 nickase, positions 1577-1586 represent a linker sequence, positions 1587-1669 represent a first occurrence of UGI, positions 1670-1679 represent a linker sequence, 1680-1762 represent a second occurrence of UGI, positions 1763-1766 represent a linker sequence, and positions 1767-1783 represent the second part of a bipartite nuclear localization sequence.

### I. CERTAIN SELECTIVITY SPECIFICATIONS OF ENGINEERED A3G BASE EDITORS

The engineered base editors provided herein enable induction of clean C>T mutations in the context of two consecutive Cs (shown as ‘C₋₁C₀’) when the target C is C₀. This has been achieved by repurposing A3G as a base editor with a series of engineered mutations that enhance A3G enzymatic activity for C-to-T conversion while maintaining its ability to accurately discriminate between adjacent Cs. The largest distribution of genetic variants associated with pathogenic mutations are point mutations (Landrum et al., 2016; Rees et al., 2018). As such, the present engineered base editors can be advantageously used in clinical applications for disease mutation correction, as exemplified below with four cases of specific DNA sequence contexts that might greatly benefit from using the present engineered A3G base editors.

A. Correcting a Pathogenic T>C Point Mutation Lying in a Coding Strand ‘DCCD’ Sequence Motif (Case 1)

In one example, there may be a T>C point mutation in the second position of a codon in the coding strand as follows: (codon 1)NND-(codon 2)CYD-(codon 3)NNN, wherein Y denotes the T>C mutation site and D represents any nucleotide other than C (i.e., A, G, or T). As such, the pathogenic T>C point mutation lies in a ‘CC’ sequence motif, wherein the target C to be corrected is the C on the right. The two consecutive Cs are located in the first and the second positions of codon 2 on the coding strand. This pathogenic mutation causes codon 2 to encode only proline (Pro) (see Table 2). Using an engineered A3G base editor of the present disclosure, all correction outcomes converge toward leucine (Leu), while using different base editors lacking the ‘CC’ preferential motif may convert the C on the left and cause mutations of amino acids other than leucine to be possible outcomes as well (Table 2). Thus, in this example, the pathogenic T>C point mutation on the DNA coding strand, which causes a Leu>Pro amino acid mutation, can be cleanly and efficiently reverted back to Leu using an engineered A3G base editor of the present disclosure.

TABLE 2
Comparison of treatment outcomes for T > C disease mutations
using A3G-BE and rAPOBEC-BE3 in the context of consecutive
‘CC’ within a codon on the coding strand.

| Possible codon 2 | | | Higher chance of |
|---|---|---|---|
| disease variants | Treatment | Possible correction outcomes | clean correction |
| CCT (Pro) | A3G-BE | CTT (Leu) | Y |
| | Others | CTT (Leu), TTT (Phe), TCT (Ser) | |
| CCA (Pro) | A3G-BE | CTA (Leu) | Y |
| | Others | TTA (Leu), CTA (Leu), TCA (Ser) | |
| CCG (Pro) | A3G-BE | CTG (Leu) | Y |
| | Others | TTG (Leu), CTG (Leu), TCG (Ser) | |

B. Correcting a Pathogenic T>C Point Mutation Lying in a Coding Strand ‘DCCCD’ Sequence Motif (Case 2)

In one example, there may be a T>C point mutation in the first position of a codon in the coding strand as follows: (codon 1)DCC-(codon 2)YDN-(codon 3)NNN, wherein Y denotes the T>C mutation site and D represents any nucleotide other than C (i.e., A, G, or T). As such, the pathogenic T>C point mutation lies in a ‘CCC’ sequence motif spanning the second and the third positions of codon 1 and the first position of codon 2, wherein the pathogenic T>C mutation occurs on the first position of codon 2. Two assumption are made: (1) All three Cs lie within the activity window of the base editor in use, and (2) the first C (the second position of codon 1 will not be mutated to T when using the engineered A3G base editors provided herein, while other base editors lacking the ‘CC’ preferential motif will or can mutate the C to T. Here, codon 2 contains only one C that causes a pathogenic variant, meaning it is easy to correct the mutation back to its original T using any C-to-T base editors. However, the immediately preceding codon (codon 1) contains ‘CC’ dinucleotide sequence on the second and third positions, making a unique case of three consecutive Cs. In order to achieve clean disease correction, only codon 2 should be corrected, leaving codon 1 unaffected by potential C-to-T byproducts. To do so, the two Cs in codon 1 should not be mutated by base editors. Similar to the Case 1 analysis, Table 3 shows that using the present engineered A3G base editors can correct the disease mutation without affecting the preceding codon by higher chance, while other base editors lacking the ‘CC’ preferential motif may generate byproducts by converting codon 1 to other non-desired amino acid sequences.

TABLE 3
Comparison of treatment outcomes for T > C disease mutations
using A3G-BE and rAPOBEC-BE3 in the context of consecutive
‘CCC’ within two codons on the coding strand.

| Disease | | | | Higher |
|---|---|---|---|---|
| Mutation | | | | chance |
| (Codon | | Possible correction outcomes | | of clean |
| 1-Codon 2) | Treatment | First a.a. | Second a.a. | correction |
| TCC-CDN | A3G-BE | TCC (Ser), TCT (Ser) | TDN or | Y |
| (Ser)-(YDN | | | CDN | |
| a.a.) | Others | TCC (Ser), TCT (Ser), | TDN or | |
| | | TTC (Phe), TTT (Phe) | CDN | |
| CCC-CDN | A3G-BE | CCC (Pro), CCT (Pro), | TDN or | Y |
| (Pro)-YDN | | CTC (Leu), CTT (Leu) | CDN | |
| a.a.) | Others | CCC (Pro), CCT (Pro), | TDN or | |
| | | CTC (Leu), TCC (Ser), | CDN | |
| | | TCT (Ser), CTT (Leu), | | |
| | | TTC (Phe), TTT (Phe) | | |
| ACC-CDN | A3G-BE | ACC (Thr), ACT (Thr) | TDN or | Y |
| (Thr)-(YDN | | | CDN | |
| a.a.) | Others | ACC (Thr), ACT (Thr), | TDN or | |
| | | ATC (Ile), ATT (Ile) | CDN | |
| GCC-CDN | A3G-BE | GCC (Ala), GCT (Ala) | TDN or | Y |
| (Ala)-(YDN | | | CDN | |
| a.a.) | Others | GCC (Ala), GCT (Ala), | TDN or | |
| | | GTC (Val), GTT (Val) | CDN | |

C. Correcting a Pathogenic T>C Point Mutation Lying in a Non-Coding Strand ‘DCCD’ Sequence Motif (Case 3)

In one example, there may be a T>C point mutation in the third position of a codon in the non-coding strand as follows: (codon 3)NNN-(codon 2)DCY-(codon 1)DNN, wherein Y denotes the T>C mutation site and D represents any nucleotide other than C (i.e., A, G, or T). As such, the pathogenic T>C mutation occurs on the non-coding strand, thereby making a ‘CC’ motif on the first and second positions of codon 2 (note: the reading direction is opposite on the non-coding strand). The possible codon 2 DNA sequences are shown in the first column of Table 4, and the corresponding complementary 5′ to 3′ DNA sequences on the coding strand are shown in the second column with each of the corresponding amino acids written in parenthesis. From the above ‘CC’ dinucleotide sequence context on the non-coding strand, all possible amino acids are glycine (Gly). For the case in which the pathogenic T>C mutation generates a ‘GCC’ on the non-coding strand (or ‘GGC’ on the coding strand), an engineered A3G base editor can cleanly mutate it into serine (Ser). The same analysis was carried out for each mutation (Table 4). In sum, pathogenic T>C mutations on the non-coding strand involving arginine Arg>Gly and Ser>Gly can be cleanly corrected back to the original Arg or Ser amino acid using the present engineered A3G base editors.

TABLE 4
Comparison of treatment outcomes for T > C disease mutations
using A3G-BE and rAPOBEC-BE3 in the context of consecutive
‘CC’ within a codon on the coding strand.

| Disease | | | | Higher chance |
|---|---|---|---|---|
| mutation | Coding | | Possible correction | of clean |
| (non-coding) | strand | Treatment | outcomes | correction |
| GCC | GGC (Gly) | A3G-BE | GCT/AGC (Ser) | Y |
| | | Others | GCT/AGC (Ser), GTC/GAC | |
| | | | (Asp), GTT/AAC (Asn) | |
| ACC | GGT (Gly) | A3G-BE | ACT/AGT (Ser) | Y |
| | | Others | ACT/AGT (Ser), ATC/GAT | |
| | | | (Asp), ATT/AAT (Asn) | |
| TCC | GGA (Gly) | A3G-BE | TCT/AGA (Arg) | Y |
| | | Others | TCT/AGA (Arg), TTC/GAA | |
| | | | (Glu), TTT/AAA (Lys) | |

D. Correcting a Pathogenic T>C Point Mutation Lying in a Non-Coding Strand ‘DCCD’ Sequence Motif (Case 4)

In one example, there may be a T>C point mutation in the second position of a codon in the non-coding strand as follows: (codon 3)NND-(codon 2)CYD-(codon 1)NNN, wherein Y denotes the T>C mutation site and D represents any nucleotide other than C (i.e., A, G, or T). As such, the ‘CC’ motif takes place on the second and third positions of codon 2 on the non-coding strand due to a pathogenic T>C mutation. The same analysis holds as Case 3. In sum, T>C disease mutations on the non-coding strand involving glutamine (Gln)>Arg and lysine (Lys)>Arg can be cleanly corrected back to the original Gln or Lys using the present engineered A3G base editors (Table 5).

TABLE 5
Comparison of treatment outcomes for T > C disease mutations using
A3G-BE and rAPOBEC-BE3 in the context of consecutive ‘CC’ within
the second and third positions of a codon on the non-coding strand.

| Disease | | | | Higher chance |
|---|---|---|---|---|
| mutation | Coding | | | of clean |
| (non-coding) | strand | Treatment | Possible correction outcomes | correction |
| CCG | CGG | A3G-BE | CTG/CAG (Gln) | Y |
| | (Arg) | Others | CTG/CAG (Gln), TCG/CGA | |
| | | | (Arg), TTG/CAA (Gln) | |
| CCA | TGG | A3G-BE | CTA/TAG (stop) | Same |
| | (Trp) | Others | CTA/TAG (stop), TCA/TGA | Same |
| | | | (stop), TTA/TAA (stop) | |
| CCT | AGG | A3G-BE | CTT/AAG (Lys) | Y |
| | (Arg) | Others | CTT/AAG (Lys), TCT/AGA | |
| | | | (Arg), TTT/AAA (Lys) | |

### II. DNA-TARGETING PROTEINS

The DNA targeting protein fused to the engineered APOBEC3G may be Streptococcus pyogenes Cas9 (SpCas9), which recognizes the NGG PAM. Alternatively the DNA targeting protein may use SunTag-mediated recruitment (Jiang et al., 2018; Tanenbaum et al., 2014). Other Cas9 variants may recognize different PAM sequence, for example Staphylococcus aureus Cas9 (SaCas9) natively recognizes the NNGRRT PAM. The DNA-targeting Cas9 protein can be replaced by other orthologs to recognize different PAM sequences to expand the targeting scope for ‘CC’ dinucleotide motif in the whole genome. These Cas9 variants include, but are not limited to, Cas9-VQR, Cas9-VRER, SaCas9, StCas9, NmCas9, CjCas9, and so on. Moreover, other native or engineered CRISPR-based DNA targeting proteins, including, but not limited to, CasX, CasY, Cpf1, C2c1, C2c2, C2c3, and others can all be fused to the engineered APOBEC3G to obtain single C-to-T conversion in the context of consecutive Cs on DNA.

Base editor variants may have expanded window sizes than the 5nt canonical window, such as by fusing a different Cas9 variant (Kim et al., 2017; Huang et al., 2019), using different recruiting systems of cytosine deaminase to the DNA target (Jiang et al., 2018), or changing lengths of guide RNAs (Ryu et al., 2018). The expanded window size in most cases increases the chance to generate non-target C-to-T byproducts when multiple Cs are present in the broadened window. In the case of an engineered variant that recognizes the ‘CC’ preferential motif, the broadened window size can increase the targeting scope for cases in which the target C within the context of consecutive ‘CC’ is positioned outside the canonical 5nt window. Conventional base editors may convert one or more of any Cs present within the broadened window while our variant in principle enables converting the single C that is within the context of ‘CC.’ However, when more than one ‘CC’ motifs are present within the broadened window, multiple Cs can still be converted and generate unwanted byproducts but less in amount than other base editors lacking the preferential motif.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a cytosine deaminase domain.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The target sequence may comprise any polynucleotide, such as DNA or RNA polynucleotides. The target sequence may be located in the nucleus or cytoplasm of the cell, such as within an organelle of the cell.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors.

Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand).

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: deaminase activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### III. BASED-EDITABLE DISEASE VARIANTS

Among the total of 1515 pathogenic SNPs identified within the BEable-GPS entries (Wang et al., 2019), 61% (929/1515) were found to lie within the CC or CNC sequence context preferred by A3G-Bes (Gehrke et al., 2018). By way of example, 540 human pathogenic SNPs were identified that could be precisely correctable by the present A3G-Bes: Familial cancer of breast, Breast-ovarian cancer, familial 1, Hereditary cancer predisposing syndrome (NM 007294.3(BRCA1):c.65T>C(p.Leu22Ser)); Cleft palate, psychomotor retardation, and distinctive facial features (NM_001009999.2(KDM1A):c.2353T>C(p.Tyr785His)); Mild non-PKU hyperphenylalaninemia (NM_000277.1(PAH):c.293T>C(p.Leu98Ser)); Amyotrophic lateral sclerosis type 1 (NM_000454.4(SOD1):c.302A>G(p.Glu101Gly)); Hemolytic anemia due to hexokinase deficiency (NM_033500.2(HK1):c.1550T>C(p.Leu517Ser)); Early infantile epileptic encephalopathy 16 (NM_001199107.1(TBC1D24):c.686T>C(p.Phe229Ser)); Hereditary diffuse leukoencephalopathy with spheroids (NM_005211.3(CSF1R):c.2483T>C(p.Phe828Ser)); TNF receptor syndrome (TRAPS)-associated periodic fever (NM_001065.3 (TNFRSF1A):c.349T>C(p.Cys117Arg)); Limb dystroglycanopathy, type C4-girdle muscular dystrophy (NM_001079802.1(FKTN):c.527T>C(p.Phe176Ser)); Alexander disease (NM_002055.4(GFAP):c.1055T>C(p.Leu352Pro)); Loeys-Dietz syndrome 2 (NM_003242.5(TGFBR2):c.923T>C(p.Leu308Pro)); Leukoencephalopathy with vanishing white matter (NM_003907.2(EIF2B5):c.1882T>C(p.Trp628Arg)); Deficiency of UDPglucose-hexo se-1-phosphateuridylyltransferase (NM_000155.3 (GALT):c.563A>G(p.Gln188Arg)); Spherocytosis, type 1, autosomal recessive (NM_000037.3(ANK1):c.-108T>C); Deafness, autosomal recessive 9 (NM_194248.2(OTOF):c.766-2A>G); Alport syndrome, X-linked recessive (NM_000495.4(COL4A5):c.1340-2A>G); WFS1-Related Disorders (NM_006005.3 (WFS1):c.2486T>C(p.Leu829Pro)); Papillon-Lefvre syndrome, Haim-Munk syndrome (NM_001814.4(CTSC):c.857A>G(p.Gln286Arg)); Congenital disorder of glycosylation type 1F (NM_004870.3(MPDU1):c.356T>C(p.Leu119Pro)); Deficiency of UDPglucose-hexose-1-phosphateuridylyltransferase (NM_000155.3(GALT):c.1138T>C(p.Ter380Arg)); Central core disease (NM_000540.2(RYR1):c.10817T>C(p.Leu3606Pro)); Schnyder crystalline corneal dystrophy (NM_013319.2(UBIAD1):c.355A>G(p.Arg119Gly)); Metachromatic leukodystrophy, adult type (NM_000487.5(ARSA):c.410T>C(p.Leu137Pro)); Osteopetrosis autosomal recessive 4 (NM_001287.5(CLCN7):c.2297T>C(p.Leu766Pro)); Zellweger syndrome (NM_000466.2(PEX1):c.1991T>C(p.Leu664Pro)); Congenital disorder of glycosylation type 1G (NM_024105.3 (ALG12):c.473T>C (p.Leu158Pro)); Acroerythrokeratoderma (NM_020427.2(SLURP1):c.43T>C (p.Trp15Arg)); Anemia, sideroblastic, pyridoxine autosomal recessive-refractory, (NM_016417.2(GLRX5):c.294A>G(p.Gln98=)); Familial hypokalemia-hypomagnesemia (NM_000339.2(SLC12A3):c.1868T>C(p.Leu623Pro)); Epidermolysis bullosa dystrophica inversa, autosomal recessive (NM_000094.3(COL7A1):c.425A>G(p.Lys142Arg)); Leprechaunism syndrome (NM_000208.2(INSR):c.779T>C(p.Leu260Pro)); Macular corneal dystrophy Type I (NM_021615.4(CHST6):c.827T>C(p.Leu276Pro)); Tyrosinemia type I (NM_000137.2(FAH):c.1141A>G(p.Arg381Gly)); X-linked agammaglobulinemia with growth hormone deficiency (NM_000061.2(BTK):c.1625T>C(p.Leu542Pro); (NM_000044.3(AR):c.2033T>C(p.Leu678Pro)); Noonan syndrome, Noonan syndrome 4, Rasopathy (NM_005633.3 (SOS1):c.1654A>G(p.Arg552Gly)); Bietti crystalline corneoretinal dystrophy (NM_207352.3(CYP4V2):c.1393A>G(p.Arg465Gly)); Pontocerebellar hypoplasia type 6 (NM_020320.3(RARS2):c.35A>G(p.Gln12Arg)); Ullrich congenital muscular dystrophy (NM_001849.3(COL6A2):c.2329T>C(p.Cys777Arg)); Deafness, autosomal recessive 7 (NM_138691.2(TMC1):c.1763+3A>G); Distal arthrogryposis type 1B (NM_002465.3(MYBPC1):c.2566T>C(p.Tyr856His)); Mitochondrial complex I deficiency (NM_016013.3 (NDUFAF1):c.758A>G(p.Lys253 Arg)); Cataract, autosomal recessive congenital 2 (NM_024513.3 (FYCO1):c.4127T>C(p.Leu1376Pro)); Distal hereditary motor neuronopathy type 5B (NM_022912.2(REEP1):c.304-2A>G); Polymicrogyria, asymmetric (NM_178012.4(TUBB2B):c.350T>C(p.Leu117Pro)); Congenital disorder of glycosylation type 1J (NM_001382.3(DPAGT1):c.503T>C(p.Leu168Pro)); Usher syndrome, type 1 (NM_000260.3(MYO7A):c.6439-2A>G); Fabry disease (NM_000169.2(GLA):c.548-2A>G); Tatton-Brown-rahman syndrome (NM_022552.4(DNMT3A):c.1943T>C(p.Leu648Pro)); Retinitis pigmentosa 51 (NM_144596.3(TTC8):c.115-2A>G); Hereditary cancer-predisposing syndrome (NM_000535.5(PMS2):c.904-2A>G); Congenital muscular hypertrophy-cerebralsyndrome (NM_006306.3 (SMC1A):c.616-2A>G); Hereditary diffuse leukoencephalopathy withspheroids (NM_005211.3 (CSF1R):c.1957T>C(p.Cys653Arg)); Hereditary diffuse leukoencephalopathy with spheroids (NM_005211.3(CSF1R):c.1745T>C(p.Leu582Pro)); Emery-Dreifuss muscular dystrophy 1, X-linked (NM_000117.2(EMD):c.266-2A>G); Niemann-Pick disease, type A, Niemann-Pickdisease, type B (NM_000543.4(SMPD1):c.475T>C(p.Cys159Arg)); not provided (NM_000169.2(GLA):c.370-2A>G); Familial hypertrophic cardiomyopathy 4, Cardiomyopathy (NM_000256.3(MYBPC3):c.1227-2A>G); Hereditary cancer-predisposing syndrome (NM_000051.3(ATM):c.3154-2A>G); Hereditary cancer-predisposing syndrome (NM_000455.4(STK11):c.545T>C(p.Leu182Pro)); Homocysteinemia due to MTHFR deficiency (NM_005957.4(MTHFR):c.388T>C(p.Cys130Arg)); Cardiac arrhythmia (NM_000238.3(KCNH2):c.1129-2A>G); Cardiac arrhythmia (NM_000238.3(KCNH2):c.2396T>C(p.Leu799Pro)); Cardiac arrhythmia (NM_000218.2(KCNQ1):c.1515-2A>G); not provided (NM_198056.2(SCN5A):c.2788-2A>G); Primary hyperoxaluria, type I (NM_000030.2(AGXT):c.777-2A>G); Primary hyperoxaluria, type III (NM_138413.3 (HOGA1):c.533T>C(p.Leu178Pro)); Epidermolysis bullosa herpetiformis, Dowling Meara (NM_000424.3 (KRT5):c.541T>C (p.Ser181Pro)); Primary hyperoxaluria, type I (NM_000030.2(AGXT):c.302T>C(p.Leu101Pro)); Citrullinemia type I (NM_000050.4(ASS1):c.421-2A>G); Ataxia-telangiectasia syndrome (NM_000051.3(ATM):c.1236-2A>G); Hereditary hemorrhagic telangiectasia type 1 (NM_000118.3(ENG):c.662T>C(p.Leu221Pro)); Hereditary factor VIII deficiency disease (NM_000132.3(F8):c.2029T>C(p.Phe677Leu)); Glycogen storage disease, type II (NM_000152.4(GAA):c.1064T>C(p.Leu355Pro)); Glycogen storage disease, type II (NM_000152.4(GAA):c.896T>C(p.Leu299Pro)); Maturity-onset diabetes of the young, type 2 (NM_000162.3(GCK):c.941T>C(p.Leu314Pro)); not provided (NM_000162.4(GCK):c.46-2A>G); Fabry disease (NM_000169.2(GLA):c.424T>C(p.Cys142Arg)); Long-chain 3-hydroxyacyl-CoA dehydrogenase deficiency (NM_000182.4(HADHA):c.1690-2A>G); not provided (NM_000199.3(SGSH):c.356-2A>G); Hereditary cancer-predisposing syndrome; Lynch syndrome (NM_000249.3(MLH1):c.883A>G(p.Ser295Gly)); Usher syndrome, type 1 (NM_000260.3(MYO7A):c.1952T>C(p.Leu651Pro)); not provided (NM_000296.3(PKD1):c.11710-2A>G); Hereditary cancer-predisposing syndrome (NM_000321.2(RB1):c.1128-2A>G); Familial hypokalemia-hypomagnesemia (NM_000339.2(SLC12A3):c.2576T>C(p.Leu859Pro)); 3-Oxo-5 alpha-steroid delta 4-dehydrogenase deficiency (NM_000348.3(SRD5A2):c.282-2A>G); Stargardt disease 1 (NM_000350.2(ABCA4):c.4462T>C(p.Cys1488Arg)); Non-immune hydrops fetalis; Sialidosis, type II (NM_000434.3(NEU1):c.353-2A>G); Familial hypercholesterolemia (NM_000527.4(LDLR):c.478T>C(p.Cys160Arg)); Li-Fraumeni syndrome (NM_000546.5(TP53):c.673-2A>G); not provided (NM_000548.4(TSC2):c.2640-2A>G); Von Hippel-Lindau syndrome (NM_000551.3(VHL):c.341-2A>G); Niemann-Pick disease, type A (NM_001007593.2(SMPD1):c.1670T>C(p.Leu557Pro)); Spastic paraplegia 74, autosomal recessive (NM_001010867.3(IBA57):c.678A>G(p.Gln226=)); Periventricular nodular heterotopia 1 (NM_001110556.1(FLNA):c.2405-2A>G); not provided (NM_001170629.1(CHD8):c.2487-2A>G); primary pulmonary hypertension (NM_001204.6(BMPR2):c.1019T>C(p.Leu340Pro)); Hereditary myopathy with early respiratory failure (NM_001267550.2(TTN):c.95185T>C(p.Trp31729Arg)); not provided (NM_001456.3(FLNA):c.2566-2A>G); Alexander's disease; not provided (NM_002055.4(GFAP):c.1096T>C(p.Tyr366His)); Alexander's disease; not provided (NM_002055.4(GFAP):c.269T>C(p.Leu90Pro)); Pulmonary fibrosis and/or bone marrow failure, telomere-related, 4 (NM_002582.3(PARN):c.246-2A>G); Cataract 30 (NM_003380.3(VIM):c.623A>G(p.Gln208Arg)); Myopia 25, autosomal dominant (NM_004199.2(P4HA2):c.419A>G(p.Gln140Arg)); Hereditary diffuse gastric cancer (NM_004360.4(CDH1):c.49-2A>G); Robinow syndrome; Robinow syndrome, autosomal dominant 3 (NM_004423.3(DVL3):c.1715-2A>G); Cystinosis (NM_004937.2(CTNS):c.853-2A>G); Methylmalonate semialdehyde dehydrogenase deficiency (NM_005589.3(ALDH6A1):c.514T>C(p.Tyr172His)); Leukodystrophy, hypomyelinating, 6 (NM_006087.3 (TUBB4A):c.1099T>C(p.Phe367Leu)); Schizophrenia (NM_014712.2(SETD1A):c.518-2A>G); Deafness, autosomal recessive 3 (NM_016239.3(MYO15A):c.6178-2A>G); Kindler's syndrome (NM_017671.4(FERMT1):c.889A>G(p.Arg297Gly)); Mucolipidosis type IV (NM_020533.2(MCOLN1):c.317T>C(p.Leu106Pro)); Familial medullary thyroid carcinoma; MEN2A and FMTC (NM_020630.4(RET):c.1825T>C(p.Cys609Arg)); Neurodevelopmental disorder with microcephaly, hypotonia, and variable brain anomalies (NM_021222.2(PRUNE1):c.521-2A>G); Joubert syndrome (NM_030578.3(B9D2):c.107T>C(p.Leu36Pro)); 3-methylglutaconic aciduria with cataracts, neurologic involvement, and neutropenia (NM_030813.5(CLPB):c.1222A>G(p.Arg408Gly)); Cone-rod dystrophy and hearing loss (NM_032171.2(CEP78):c.1629-2A>G); Bardet-Biedl syndrome 4 (NM_033028.4(BBS4):c.406-2A>G); Leigh syndrome due to mitochondrial complex I deficiency (NM_152416.3(NDUFAF6):c.820A>G(p.Arg274Gly)); not provided (NM_173483.3(CYP4F22):c.1007-2A>G); DICER1-related pleuropulmonary blastoma cancer predisposition syndrome (NM_177438.2(DICER1):c.2236A>G(p.Arg746Gly)); Deafness, autosomal recessive 9 (NM_194248.2(OTOF):c.5992T>C(p.Ter1998Arg)); Epileptic encephalopathy, early infantile, 53 (NM_203446.2(SYNJ1):c.3365-2A>G); Congenital long QT syndrome (NM_000335.4(SCN5A):c.3971A>G(p.Asn1324Ser)); Epidermolysis bullosa herpetiformis, Dowling Meara (NM_000526.4(KRT14):c.368A>G(p.Asn123Ser)); Pachyonychia congenita type 1, Palmoplantar keratoderma, nonepidermolytic, focal (NM_005557.3(KRT16):c.374A>G(p.Asn125Ser)); Hyperkalemic Periodic Paralysis Type 1, Paramyotonia congenita of von Eulenburg (NM_000334.4(SCN4A):c.2078T>C(p.Ile693Thr)); Hyperkalemic Periodic Paralysis Type 1 (NM_000334.4(SCN4A):c.4078A>G(p.Met1360Val)); Hyperkalemic Periodic Paralysis Type 1 (NM_000334.4(SCN4A):c.4108A>G(p.Met1370Val)); Familial hyperkalemic periodic paralysis e 1 (NM_000334.4(SCN4A):c.4774A>G(p.Met1592Val)); Primary erythromelalgia (NM_002977.3(SCN9A):c.2543T>C(p.Ile848Thr)); Immunodeficiency 17 (NM_000073.2(CD3G):c.1A>G(p.Met1Val)); Papillon Periodontitis, aggressive, Papillon-Lefvre syndrome (NM_001814.4(CTSC):c.1040A>G(p.Tyr347Cys)); Islet cell hyperplasia (NM_000525.3(KCNJ11):c.776A>G(p.His259Arg)); Deficiency of UDPglucose-hexose-1-phosphateuridylyltransferase (NM_000155.3 (GALT):c.386T>C (p.Met129Thr)); Pontocerebellar hypoplasia type 2B (NM_025265.3(TSEN2):c.926A>G(p.Tyr309Cys)); Malignant hyperthermia susceptibility type 1, Central core disease (NM_000540.2(RYR1):c.14693T>C(p.Ile4898Thr)); Lethal arthrogryposis with anterior horn cell disease (NM_001003722.1(GLE1):c.2051T>C(p.Ile684Thr)); Spastic paraplegia 10 (NM_004984.2(KIF5A):c.827A>G(p.Tyr276Cys) (m.4269A>G)); Skeletal defects, genital hypoplasia, and mentalretardation (NM_006006.4(ZBTB16):c.1849A>G(p.Met617Val)); Eichsfeld type congenital muscular dystrophy (NM_020451.2(SEPN1):c.1A>G(p.Met1Val)); Ehlers-Danlos syndrome, musculocontractural type (NM_130468.3(CHST14):c.878A>G(p.Tyr293Cys)); Amyotrophic lateral sclerosis type 1 (NM_000454.4(SOD1):c.341T>C(p.Ile114Thr)); Fibrosis of extraocular muscles, congenital, 1 (NM_001173464.1(KIF21A):c.2839A>G(p.Met947Val)); Factor xiii, a subunit, deficiency of (NM_000129.3(F13A1):c.851A>G(p.Tyr284Cys)); Thyroid hormone resistance, generalized, autosomal dominant (NM_001128177.1(THRB):c.1324A>G(p.Met442Val)); Hereditary factor IX deficiency disease (NM_000133.3(F9):c.917A>G(p.Asn306Ser)); Hereditary factor VIII deficiency disease (NM_000132.3(F8):c.398A>G(p.Tyr133Cys)); Distal hereditary motor neuronopathy type 5, Silver spastic paraplegia syndrome, CharcotMarie-Tooth disease, type 2 (NM_032667.6(BSCL2):c.263A>G(p.Asn88Ser)); Incontinentia pigmenti syndrome (NM_003639.4(IKBKG):c.1219A>G(p.Met407Val)); Marfan syndrome (NM_000138.4(FBN1):c.4987T>C(p.Cys1663Arg)); Emery-Dreifuss muscular dystrophy, X-linked (NM_000117.2(EMD):c.1A>G(p.Met1Val)); Dursun syndrome (NM_138387.3(G6PC3):c.346A>G(p.Met116Val)); Hereditary Nonpolyposis Colorectal Neoplasms (NM_000249.3(MLH1):c.884+4A>G); Parkinson disease 8, autosomal dominant (NM_198578.3(LRRK2):c.5605A>G(p.Met1869Val)); not provided (NM_002693.2(POLG):c.1808T>C(p.Met603Thr)); not provided (NM_174917.4(ACSF3):c.1A>G(p.Met1Val)); Polyarteritis nodosa (NM_001282227.1(CECR1):c.1232A>G(p.Tyr411Cys)); Hereditary factor IX deficiency disease (NM_000133.3(F9):c.1031T>C(p.Ile344Thr)); Severe congenital neutropenia X-linked (NM_000377.2(WAS):c.881T>C(p.Ile294Thr)); Osteopetrosis autosomal dominant type 2, Osteopetrosis autosomal recessive 4 (NM_001287.5(CLCN7):c.296A>G(p.Tyr99Cys)); Cystic fibrosis (NM_000492.3(CFTR):c.1A>G(p.Met1Val)); Biotinidase deficiency (NM_000060.3(BTD):c.278A>G(p.Tyr93Cys)); Biotinidase deficiency (NM_000060.3(BTD):c.1313A>G(p.Tyr438Cys)); Early infantile epileptic encephalopathy 7 (NM_172107.2(KCNQ2):c.1636A>G(p.Met546Val)); Congenital contractural arachnodactyly (NM_001999.3(FBN2):c.3725-15A>G); Atrial fibrillation, familial, 16 (NM_018400.3 (SCN3B):c.482T>C(p.Met161Thr)); Myopia 24, autosomal dominant (NM_173596.2(SLC39A5):c.911T>C(p.Met304Thr)); X-linked hereditary motor and sensory neuropathy (NM_000166.5(GJB1):c.580A>G(p.Met194Val)); Charcot-Marie-Tooth disease, X-linked recessive, type 5, Deafness, high-frequency sensorineural, X-linked (NM_002764.3(PRPS1):c.343A>G(p.Met115Val)); Episodic pain syndrome, familial, 3 (NM_001287223.1(SCN11A):c.1142T>C(p.Ile381Thr)); Cardiomyopathy (NM_000257.3(MYH7):c.1615A>G(p.Met539Val)); Spondyloepimetaphyseal dysplasia with joint laxity (NM_080605.3(B3GALT6):c.1A>G(p.Met1Val)); Thoracic aortic aneurysms and aortic dissections (NM_000138.4(FBN1):c.7916A>G(p.Tyr2639Cys)); not provided (NM_000255.3(MUT):c.329A>G(p.Tyr110Cys)); Transposition of great arteries (NM_015335.4(MED13L):c.6068A>G(p.Asp2023Gly)); not provided (NM_000083.2(CLCN1):c.1453A>G(p.Met485Val)); not provided (NM_000137.2(FAH):c.1A>G(p.Met1Val)); Deficiency of UDPglucose-hexose-1-phosphate uridylyltransferase (NM_000155.3 (GALT):c.425T>C(p.Met142Thr)); Epidermolytic palmoplantar keratoderma (NM_000226.3(KRT9):c.470T>C(p.Met157Thr)); not provided (NM_000238.3(KCNH2):c.125T>C(p.Ile42Thr)); Familial hypertrophic cardiomyopathy 1; Hypertrophic cardiomyopathy; Primary familial hypertrophic cardiomyopathy (NM_000257.3(MYH7):c.2207T>C(p.Ile736Thr)); Deafness, autosomal recessive 2 (NM_000260.3(MYO7A):c.620A>G(p.Asn207Ser)); not provided (NM_000267.3(NF1):c.2T>C(p.Met1Thr)); not provided (NM_000277.2(PAH):c.1249T>C(p.Tyr417His)); not provided (NM_000277.2(PAH):c.2T>C(p.Met1Thr)); Aniridia 1 (NM_000280.4(PAX6):c.1A>G(p.Met1Val)); not provided (NM_000424.3(KRT5):c.1A>G(p.Met1Val)); Enlarged vestibular aqueduct; Pendred's syndrome (NM_000441.1(SLC26A4):c.2T>C(p.Met1Thr)); Metachromatic leukodystrophy (NM_000487.5(ARSA):c.674A>G(p.Tyr225Cys)); Tay-Sachs disease; not provided (NM_000520.5(HEXA):c.2T>C(p.Met1Thr)); Familial hypercholesterolemia (NM_000527.4(LDLR):c.248T>C(p.Ile83Thr)); Maturity-onset diabetes of the young, type 3 (NM_000545.6(HNF1A):c.1A>G(p.Met1Val)); Pseudoxanthoma elasticum (NM_001171.5(ABCC6):c.3380T>C(p.Met1127Thr)); Cortical dysplasia, complex, with other brain malformations 1 (NM_001197181.1(TUBB3):c.751A>G(p.Met251Val)); Cortical dysplasia, complex, with other brain malformations 1 (NM_001197181.1(TUBB3):c.946A>G(p.Met316Val)); Osteopetrosis autosomal dominant type 2; Osteopetrosis autosomal recessive 4 (NM_001287.5(CLCN7):c.296A>G(p.Tyr99Cys)); Bleeding disorder, platelet-type, 21 (NM_002017.4(FLI1):c.1028A>G(p.Tyr343Cys)); Generalized epilepsy and paroxysmal dyskinesia (NM_002247.3(KCNMA1):c.2984A>G(p.Asn995Ser)); Spastic paraplegia 7 (NM_003119.3(SPG7):c.2228T>C(p.Ile743Thr)); Mitochondrial short-chain enoyl-coa hydratase 1 deficiency (NM_004092.3(ECHS1):c.176A>G(p.Asn59Ser)); not provided (NM_004321.7(KIF1A):c.1040A>G(p.Tyr347Cys)); not provided (NM_004369.3(COL6A3):c.6309+3A>G); Rubinstein-Taybi syndrome (NM_004380.2(CREBBP):c.5614A>G(p.Met1872Val)); Glycogen storage disease, type V (NM_005609.3(PYGM):c.1A>G(p.Met1Val)); Leukodystrophy, hypomyelinating, 6 (NM_006087.3(TUBB4A):c.1162A>G(p.Met388Val)); Epileptic encephalopathy, early infantile, 58 (NM_006180.4(NTRK2):c.1301A>G(p.Tyr434Cys)); Craniosynostosis 4 (NM_006494.3(ERF):c.1A>G(p.Met1Val)); Spinocerebellar ataxia 28 (NM_006796.2(AFG3L2):c.1997T>C(p.Met666Thr)); Helix syndrome (NM_006984.4(CLDN10):c.2T>C(p.Met1Thr)); Pseudo hypoaldosteronism, type 2 (NM_017415.2(KLHL3):c.232A>G(p.Met78Val)); Combined oxidative phosphorylation deficiency 26 (NM_020810.3(TRMT5):c.1156A>G(p.Met386Val)); not provided (NM_022168.3(IFIH1):c.1328A>G(p.Asp443Gly)); Blepharophimosis, ptosis, and epicanthus inversus (NM_023067.3(FOXL2):c.644A>G(p.Tyr215Cys)); Brown-Vialetto-Van Laere syndrome 1 (NM_033409.3(SLC52A3):c.224T>C(p.Ile75Thr)); Methylmalonic aciduria cblB type (NM_052845.3(MMAB):c.2T>C(p.Met1Thr)); 4-Alpha-hydroxyphenylpyruvate hydroxyladeficiency (NM_002150.2(HPD):c.97G>A(p.Ala33Thr)); Phenylketonuria, Hyperphenylalaninemia, nonpku (NM_000277.1(PAH):c.1241A>G(p.Tyr414Cys)); Leber congenital amaurosis 13 (NM_152443.2(RDH12):c.677A>G(p.Tyr226Cys)); alpha Thalassemia (NM_000518.4(HBB):c.59A>G(p.Asn20Ser)); Juvenile retinoschisis (NM_000330.3(RS1):c.286T>C(p.Trp96Arg)); Deficiency of UDPglucose-hexose-1-phosphateuridylyltransferase (NM_000155.3(GALT):c.290A>G(p.Asn97Ser)); Brown-Vialetto-Van laere syndrome (NM_033409.3(SLC52A3):c.1238T>C(p.Val413A1a)); Primary pulmonary hypertension (NM_000020.2(ACVRL1):c.293A>G(p.Asn98Ser)); Congenital long QT syndrome (NM_000238.3(KCNH2):c.1886A>G(p.Asn629Ser)); MYH-associated polyposis (NM_001128425.1(MUTYH):c.713A>G(p.Asn238Ser)); Porokeratosis 7, multiple types (NM_002461.2(MVD):c.875A>G(p.Asn292Ser)); Lissencephaly 3 (NM_006009.3(TUBA1A):c.1226T>C(p.Val409Ala)); Hereditary Nonpolyposis Colorectal Neoplasms (NM_000179.2(MSH6):c.1346T>C(p.Leu449Pro; NM_000041.3(APOE):c.137T>C(p.Leu46Pro)); Maturity-onset diabetes of the young, type 3 (NM_000545.6(HNF1A):c.1720G>A(p.Gly574Ser)); Ischiopatellar dysplasia (NM_018488.2(TBX4):c.1592A>G(p.Gln531Arg)); Immunoglobulin A deficiency 2, Common variable immunodeficiency 2 (NM_012452.2(TNFRSF13B):c.310T>C(p.Cys104Arg)); Beta thalassemia intermedia (NM_000518.4(HBB):c.344T>C(p.Leu115Pro)); Stargardt disease, Cone rod dystrophy 3 (NM_000350.2(ABCA4):c.5819T>C(p.Leu1940Pro)); not provided (NM_000531.5(OTC):c.988A>G(p.Arg330Gly)); Anonychia (NM_001029871.3(RSPO4):c.194A>G(p.Gln65Arg)); Hypogonadotropic hypogonadism 17 with or without anosmia (NM_030964.3(SPRY4):c.530A>G(p.Lys177Arg)); Glucose-6-phosphate transport defect (NM_001164277.1(SLC37A4):c.352T>C(p.Trp118Arg)); Arthrogryposis multiplex congenita, distal, X linked (NM_003334.3(UBA1):c.1639A>G(p.Ser547Gly)); Deafness, autosomal recessive 9 (NM_194248.2(OTOF):c.3032T>C(p.Leu1011Pro)); Familial cancer of breast, Breast-ovarian cancer, familial 1 (NM_007294.3(BRCA1):c.5291T>C(p.Leu1764Pro)); Lattice corneal dystrophy Type III (NM_002353.2(TACSTD2):c.557T>C(p.Leu186Pro)); Familial cancer of breast, Breast cancer, familial 2-ovarian (NM_000059.3(BRCA2):c.7958T>C(p.Leu2653Pro)); Ganglioside sialidase deficiency (NM_020533.2(MCOLN1):c.406-2A>G); Smith-Magenis syndrome (NM_030665.3(RAI1):c.4685A>G(p.Gln1562Arg)); Danon disease (NM_001122606.1(LAMP2):c.961T>C(p.Trp321Arg)); Allan-Herndon-Dudley syndrome (NM_006517.4(SLC16A2):c.1313T>C(p.Leu438Pro)); Juvenile retinoschisis (NM_000330.3(RS1):c.38T>C(p.Leu13Pro)); TNF receptor syndrome (TRAPS)-associated periodic fever (NM_001065.3(TNFRSF1A):c.175T>C(p.Cys59Arg)); Usher syndrome, type 1 (NM_000260.3(MYO7A):c.1344-2A>G); Hereditary pancreatitis (NM_002769.4(PRSS1):c.68A>G(p.Lys23Arg)); 46, XY gonadal dysgenesis, complete, DHH-related (NM_021044.2(DHH):c.485T>C(p.Leu162Pro)); X-linked severe combined immunodeficiency (NM_000206.2(IL2RG):c.343T>C(p.Cys115Arg)); Deficiency of UDPglucose-hexose-1-phosphateuridylyltransferase (NM_000155.3(GALT):c.677T>C(p.Leu226Pro)); Cystinosis (NM_004937.2(CTNS):c.473T>C(p.Leu158Pro)); Mitochondrial complex I deficiency (NM_025152.2(NUBPL):c.815-27T>C); Hyperlipoproteinemia, type I (NM_000237.2(LPL):c.337T>C(p.Trp113Arg)); Holocarboxylase synthetase deficiency (NM_000411.6(HLCS):c.710T>C(p.Leu237Pro)); Congenital disorder of glycosylation type 1D (NM_005787.5(ALG3):c.211T>C(p.Trp71Arg)); Marinesco-Sjögren syndrome (NM_001037633.1(SIL1):c.1370T>C(p.Leu457Pro)); Isovaleric acidemia, type I (NM_002225.3(IVD):c.134T>C(p.Leu45Pro)); AICAR transformylase/IMP cyclohydrolase deficiency (NM_004044.6(ATIC):c.1277A>G(p.Lys426Arg)); Neoplasm of stomach (NM_001128425.1(MUTYH):c.1241A>G(p.Gln414Arg)); Primary hyperoxaluria, type I (NM_000030.2(AGXT):c.613T>C(p.Ser205Pro)); Spondyloepiphyseal dysplasia with congenital joint dislocations (NM_004273.4(CHST3):c.920T>C(p.Leu307Pro)); Osteopetrosis autosomal recessive 7 (NM_003839.3(TNFRSF11A):c.508A>G(p.Arg170Gly)); Severe combined immunodeficiency due to ADA deficiency (NM_000022.2(ADA):c.320T>C(p.Leu107Pro)); Progressive pseudo rheumatoid dysplasia (NM_003880.3(WISP3):c.232T>C(p.Cys78Arg)); Generalized epilepsy with febri type 7, not specified le seizures plus (NM_002977.3(SCN9A):c.1964A>G(p.Lys655Arg)); Centromeric instability of chromosomes 1, 9 and 16 and immunodeficiency (NM_006892.3(DNMT3B):c.808T>C(p.Ser270Pro)); Bartter syndrome type 3 (NM_000085.4(CLCNKB):c.1294T>C(p.Tyr432His); (NM_001300.5(KLF6):c.506T>C(p.Leu169Pro)); Acid-labile subunit deficiency (NM_004970.2(IGFALS):c.1618T>C(p.Cys540Arg)); Nemaline myopathy 3 (NM_001100.3(ACTA1):c.287T>C(p.Leu96Pro)); Glucocorticoid resistance, generalized (NM_001018077.1(NR3C1):c.2209T>C(p.Phe737Leu)); Pseudohypoaldosteronism type 1 autosomal (NM_000901.4(NR3C2):c.2327A>G(p.Gln776Arg)); Sucrase-isomaltase deficiency (NM_001041.3(SI):c.1859T>C(p.Leu620Pro)); Oculocutaneous albinism type 4 (NM_016180.4(SLC45A2):c.1082T>C(p.Leu361Pro)); Merosin deficient congenital muscular dystrophy (NM_000426.3(LAMA2):c.7691T>C(p.Leu2564Pro)); Bile acid malabsorption, primary (NM_000452.2(SLC10A2):c.728T>C(p.Leu243Pro)); Infantile hypophosphatasia (NM_000478.4(ALPL):c.1306T>C(p.Tyr436His)); Infantile hypophosphatasia (NM_000478.4(ALPL):c.979T>C(p.Phe327Leu)); Gaucher disease, atypical, due to saposin C deficiency (NM_001042465.1(PSAP):c.1055T>C(p.Leu352Pro)); Spinocerebellar ataxia 5 (NM_006946.2(SPTBN2):c.758T>C(p.Leu253Pro)); Hereditary pyro poikilocytosis, Elliptocytosis 2 (NM_003126.2(SPTA1):c.620T>C(p.Leu207Pro)); Methemoglobinemia type 1 (NM_000398.6(CYB5R3):c.446T>C(p.Leu149Pro)); X-linked agammaglobulinemia (NM_000061.2(BTK):c.1223T>C(p.Leu408Pro)); Severe congenital neutropenia X-linked (NM_000377.2(WAS):c.809T>C(p.Leu270Pro)); Glycogen storage disease, type IV (NM_000158.3(GBE1):c.671T>C(p.Leu224Pro)); Glanzmann thrombasthenia (NM_000419.3(ITGA2B):c.641T>C(p.Leu214Pro)); Retinitis pigmentosa 46 (NM_006899.3(IDH3B):c.395T>C(p.Leu132Pro)); Desbuquois syndrome (NM_001159772.1(CANT1):c.671T>C(p.Leu224Pro)); Primary hyperoxaluria, type I (NM_000030.2(AGXT):c.449T>C(p.Leu150Pro)); Primary hyperoxaluria, type I (NM_000030.2(AGXT):c.757T>C(p.Cys253Arg)); Primary hyperoxaluria, type I (NM_000030.2(AGXT):c.893T>C(p.Leu298Pro)); Sialidosis, type II (NM_000434.3(NEU1):c.1088T>C(p.Leu363Pro)); Permanent neonatal diabetes mellitus (NM_000352.4(ABCC8):c.404T>C(p.Leu135Pro)); Dyskeratosis congenita X-linked (NM_001363.4(DKC1):c.941A>G(p.Lys314Arg)); Gorlin syndrome, Holoprosencephaly 7 (NM_000264.3 (PTCH1):c.2479A>G(p.Ser827Gly)); Cytochrome-c oxidase deficiency, Mitochondrial complex I deficiency (m.5728T>C); Hemolytic anemia, nonspherocytic, due to glucose phosphate isomerase deficiency (NM_000175.3(GPI):c.1028A>G(p.Gln343Arg)); Myoclonic epilepsy, familial infantile (NM_001199107.1(TBC1D24):c.751T>C(p.Phe251Leu)); Norum disease (NM_000229.1(LCAT):c.508T>C(p.Trp170Arg)); Early infantile epileptic encephalopathy 2 (NM_003159.2(CDKL5):c.659T>C(p.Leu220Pro)); Joubert syndrome 13 (NM_024549.5(TCTN1):c.221-2A>G); Leigh disease (NC 012920.1:m.5559A>G); Primary familial hypertrophic cardiomyopathy, Familial hypertrophic cardiomyopathy 4, Cardiomyopathy (NM_000256.3 (MYBPC3):c.26-2A>G); Nephronophthisis 16 (NM_173551.4(ANKS6):c.1322A>G(p.Gln441Arg)); Cystic fibrosis (NM_000492.3(CFTR):c.3717+4A>G); Aortic aneurysm, familial thoracic 4 (NM_001040113.1(MYH11):c.3791T>C(p.Leu1264Pro)); Peroxisome biogenesis disorder 4B (NM_000287.3(PEX6):c.1601T>C(p.Leu534Pro)); Preeclampsia/eclampsia 5 (NM_006587.3(CORIN):c.1414A>G(p.Ser472Gly); (NM_004453.3(ETFDH):c.1130T>C(p.Leu377Pro)); Pityriasis rubra pilaris (NM_024110.4(CARD14):c.467T>C(p.Leu156Pro)); Short stature, onychodysplasia, facial dysmorphism, and hypotrichosis (NM_001161581.1(POC1A):c.398T>C(p.Leu133Pro)); Porokeratosis, disseminated superficial actinicl (NM_000431.3(MVK):c.122T>C(p.Leu41Pro)); Deafness, autosomal recessive 89 (NM_001130089.1(KARS):c.517T>C(p.Tyr173His)); Dyschromatosis universalis hereditaria 3 (NM_005689.2(ABCB6):c.508A>G(p.Ser170Gly)); Permanent neonatal diabetes mellitus (NM_000207.2(INS):c.*59A>G); Deafness, autosomal recessive 9 (NM_194248.2(OTOF):c.3413T>C(p.Leu1138Pro)); Familial hypertrophic cardiomyopathy 4, Cardiomyopathy (NM_000256.3(MYBPC3):c.1224-2A>G); Parkinson disease 19, juvenile-onset (NM_001256864.1(DNAJC6):c.801-2A>G); Hypobetalipoproteinemia, familial, 2 (NM_014495.3(ANGPTL3):c.883T>C(p.Phe295Leu)); Kabuki make-up syndrome (NM_003482.3(KMT2D):c.5645-2A>G); Glycogen storage disease type 1A (NM_000151.3(G6PC):c.230+4A>G); Enamel-renal syndrome (NM_017565.3(FAM20A):c.590-2A>G); Activated PI3K-delta syndrome (NM_005026.3(PIK3CD):c.1246T>C(p.Cys416Arg)); Sialic acid storage disease, severe infantile type (NM_012434.4(SLC17A5):c.500T>C(p.Leu167Pro)); not provided (NM_003159.2(CDKL5):c.602T>C(p.Leu201Pro)); Heterotopia (NM_178151.2(DCX):c.683T>C(p.Leu228Pro)); Severe X-linked myotubular myopathy (NM_000252.2(MTM1):c.550A>G(p.Arg184Gly)); Orofaciodigital syndrome 6 (NM_023073.3(C5orf42):c.3290-2A>G); Hereditary diffuse leukoencephalopathy with spheroids (NM_005211.3(CSF1R):c.2655-2A>G); Familial hypertrophic cardiomyopathy 4 (NM_000256.3(MYBPC3):c.2906-2A>G); Familial hypercholesterolemia (NM_000527.4(LDLR):c.1468T>C(p.Trp490Arg)); Cardiomyopathy (NM_000257.3(MYH7):c.617A>G(p.Lys206Arg)); Rasopathy (NM_002880.3(RAF1):c.1279A>G(p.Ser427Gly)); Limb-girdle muscular dystrophy, type 2B (NM_003494.3(DYSF):c.1285-2A>G); Achromatopsia 5 (NM_006204.3(PDE6C):c.1483-2A>G); Retinitis pigmentosa 71 (NM_015662.2(IFT172):c.770T>C(p.Leu257Pro)); Congenital disorder of glycosylation type 1K (NM_019109.4(ALG1):c.1188-2A>G); not provided (NM_172107.2(KCNQ2):c.848A>G(p.Lys283Arg)); not provided (NM_014191.3(SCN8A):c.4889T>C(p.Leu1630Pro)); not provided (NM_000391.3(TPP1):c.833A>G(p.Gln278Arg)); Leukoencephalopathy with vanishing white matter, Ovarioleukodystrophy (NM_014239.3(EIF2B2):c.638A>G(p.Glu213Gly)); Primary hyperoxaluria, type I (NM_000030.2(AGXT):c.1076T>C(p.Leu359Pro)); Deafness, nonsyndromic sensorineural, mitochondrial (m.7443A>G); Pulmonary arterial hypertension related to hereditary hemorrhagic telangiectasia (NM_000020.2(ACVRL1):c.1195T>C(p.Trp399Arg)); not provided (NM_000070.2(CAPN3):c.2185-2A>G); not provided (NM_000101.3(CYBA):c.155T>C(p.Leu52Pro)); not provided (NM_000138.4(FBN1):c.6772T>C(p.Cys2258Arg)); not provided (NM_000138.4(FBN1):c.6872-2A>G); Hereditary cancer-predisposing syndrome; Lynch syndrome (NM_000179.2(MSH6):c.3632T>C(p.Leu1211Pro)); not provided (NM_000193.3(SHH):c.592T>C(p.Cys198Arg)); not provided (NM_000199.3(SGSH):c.1139A>G(p.Gln380Arg)); Mucopolysaccharidosis, MPS-III-A (NM_000199.3(SGSH):c.673T>C(p.Phe225Leu)); Hereditary cancer-predisposing syndrome; Lynch syndrome (NM_000249.3(MLH1):c.2246T>C(p.Leu749Pro)); Methylmalonic aciduria due to methyl malonyl-CoA mutase deficiency (NM_000255.3(MUT):c.1853T>C(p.Leu618Pro)); Cardiovascular phenotype; Hypertrophic cardiomyopathy; Primary familial hypertrophic cardiomyopathy (NM_000256.3(MYBPC3):c.2309-2A>G); not provided (NM_000264.4(PTCH1):c.1348-2A>G); not provided (NM_000267.3(NF1):c.4270-2A>G); not provided (NM_000359.2(TGM1):c.877-2A>G); Polyglandular autoimmune syndrome, type 1 (NM_000383.3(AIRE):c.232T>C(p.Trp78Arg)); Sphingomyelin/cholesterol lipidosis, Niemann Pick disease, type A, Niemann-Pick disease, type B (NM_000543.4(SMPD1):c.911T>C(p.Leu304Pro)); not provided (NM_000435.2(NOTCH3):c.1597T>C(p.Cys533Arg)); Retinitis pigmentosa 43 (NM_000440.2(PDE6A):c.1408-2A>G); alpha Thalassemia; not specified (NM_000517.4(HBA2):c.*94A>G); Hemolytic anemia (NM_000518.4(HBB):c.127T>C(p.Phe43Leu)); Familial hypercholesterolemia (NM_000527.4(LDLR):c.1102T>C(p.Cys368Arg)); Familial hypercholesterolemia (NM_000527.4(LDLR):c.1154T>C(p.Leu385Pro)); Familial hypercholesterolemia (NM_000527.4(LDLR):c.1207T>C(p.Phe403Leu)); Familial hypercholesterolemia (NM_000527.4(LDLR):c.1468T>C(p.Trp490Arg)); Familial hypercholesterolemia (NM_000527.4(LDLR):c.166T>C(p.Ser56Pro)); Familial hypercholesterolemia (NM_000527.4(LDLR):c.382T>C(p.Cys128Arg)); Familial hypercholesterolemia (NM_000527.4(LDLR):c.691T>C(p.Cys231Arg)); Familial hypercholesterolemia (NM_000527.4(LDLR):c.985T>C(p.Cys329Arg)); Hereditary cancer-predisposing syndrome; Li-Fraumeni syndrome (NM_000546.5(TP53):c.920-2A>G); Tuberous sclerosis syndrome (NM_000548.4(TSC2):c.226-2A>G); Tuberous sclerosis syndrome (NM_000548.4(TSC2):c.2546-2A>G); not provided; von Willebrand disease, type 2a (NM_000552.4(VWF):c.3814T>C(p.Cys1272Arg)); Short-rib polydactyly syndrome type III (NM_001080463.1(DYNC2H1):c.6866T>C(p.Leu2289Pro)); Joubert syndrome 9 (NM_001080522.2(CC2D2A):c.1676T>C(p.Leu559Pro)); Abnormality of neuronal migration (NM_001083961.1(WDR62):c.2030T>C(p.Leu677Pro)); Patent ductus arteriosus 3 (NM_001136239.3(PRDM6):c.1385A>G(p.Gln462Arg)); Encephalopathy, neonatal severe, with lactic acidosis and brain abnormalities (NM_001144869.2(LIPT2):c.89T>C(p.Leu30Pro)); Pseudoxanthoma elasticum (NM_001171.5(ABCC6):c.1192A>G(p.Ser398Gly)); Pseudoxanthoma elasticum (NM_001171.5(ABCC6):c.2018T>C(p.Leu673Pro)); Pseudoxanthoma elasticum (NM_001171.5(ABCC6):c.3715T>C(p.Tyr1239His)); Pseudoxanthoma elasticum (NM_001171.5(AB CC6):c.601-2A>G); Digitorenocerebral syndrome (NM_001199107.1(TBC1D24):c.313T>C(p.Cys105Arg)); Mitochondrial DNA depletion syndrome 1 (MNGIE type) (NM_001257988.1(TYMP):c.1112T>C(p.Leu371Pro)); leukodystrophy, progressive, early childhood-onset (NM_001300953.1(ACER3):c.98A>G(p.Glu33Gly)); Cirrhosis, cryptogenic (NM_002273.3(KRT8):c.160T>C(p.Tyr54His)); not provided (NM_002294.2(LAMP2):c.929-2A>G); Congenital cystic disease of liver (NM_002743.3 (PRKCSH):c.1341-2A>G); Neurodegeneration with brain iron accumulation 2b (NM_003560.3(PLA2G6):c.1349-2A>G); Glycosylphosphatidylinositol biosynthesis defect 15 (NM_003801.3(GPAA1):c.869T>C(p.Leu290Pro)); Glycosylphosphatidylinositol biosynthesis defect 15 (NM_003801.3(GPAA1):c.872T>C(p.Leu291Pro)); Mitochondrial short-chain enoyl-coa hydratase 1 deficiency; not provided (NM_004092.3(ECHS1):c.476A>G(p.Gln159Arg)); Focal cortical dysplasia type II (NM_004958.3(MTOR):c.7280T>C(p.Leu2427Pro)); Chromosome Xq28 deletion syndrome (NM_005745.7(BCAP31):c.194-2A>G); Senior-Loken syndrome 7 (NM_006642.4(SDCCAG8):c.221-2A>G); Mental retardation, autosomal dominant 5 (NM_006772.2(SYNGAP1):c.388-2A>G); Epilepsy, early-onset, vitamin b6-dependent (NM_007198.3(PLPBP):c.320-2A>G); Hereditary breast and ovarian cancer syndrome (NM_007294.3(BRCA1):c.4186-2A>G); Breast-ovarian cancer, familial 1 (NM_007294.3(BRCA1):c.81-2A>G); Neurodevelopmental disorder with or without hyperkinetic movements and seizures, autosomal dominant (NM_007327.3(GRIN1):c.2449T>C(p.Phe817Leu)); not provided (NM_015284.3(SZT2):c.8655-2A>G); Anosmia; Arhinia choanal atresia microphthalmia (NM_015295.2(SMCHD1):c.1034A>G(p.Gln345Arg)); Joubert syndrome (NM_019892.5(INPP5E):c.1684A>G(p.Ser562Gly)); MEN2 phenotype: Unclassified (NM_020630.4(RET):c.1831T>C(p.Cys611Arg)); Posterior polymorphous corneal dystrophy 1 (NM_021220.3(OVOL2):c.-370T>C); Joubert syndrome 5; Meckel syndrome type 4 (NM_025114.3(CEP290):c.181-2A>G); Inborn genetic diseases; Mitochondrial complex I deficiency (NM_025152.2(NUBPL):c.311T>C(p.Leu104Pro)); Pseudo hypoaldosteronism type 2B (NM_032387.4(WNK4):c.1679A>G(p.Glu560Gly)); Congenital generalized lipodystrophy type 2 (NM_032667.6(BSCL2):c.672-2A>G); Verheij syndrome (NM_078480.2(PUF60):c.1381-2A>G); not provided (NM_144997.5(FLCN):c.1177-2A>G); Early infantile epileptic encephalopathy 7 (NM_172107.3(KCNQ2):c.1024-2A>G); Early infantile epileptic encephalopathy 7 (NM_172107.3(KCNQ2):c.1203T>C(p.Ser401=)); Spondyloepiphyseal dysplasia; Spondyloepiphyseal dysplasia—Sutcliffe type (NM_212482.2(FN1):c.367T>C(p.Cys123Arg)); Pyridoxal 5-phosphate-dependent epilepsy (NM_018129.3 (PNPO):c.784T>C(p.Ter262Gln)); Mucopolysaccharidosis, MPS-II (NM_000202.6(IDS):c.404A>G(p.Lys135Arg)); Dyskeratosis congenita autosomal recessive 1, Dyskeratosis congenita, autosomal recessive 2 (NM_017838.3(NHP2):c.415T>C(p.Tyr139His)); Idiopathic fibro sing alveolitis, chronic form (NM_001098668.2(SFTPA2):c.593T>C(p.Phe198Ser)); Isolated growth hormone deficiency type 1B (NM_000823.3(GHRHR):c.985A>G(p.Lys329Glu)); Congenital myopathy with fiber type disproportion (NM_152263.3(TPM3):c.505A>G(p.Lys169Glu)); Axenfeld-Rieger syndrome type 1 (NM_153427.2(PITX2):c.262A>G(p.Lys88Glu)); Intrauterine growth retardation, metaphyseal dysplasia, adrenal hypoplasia congenita, and genital anomalies (NM_000076.2(CDKN1C):c.832A>G(p.Lys278 Glu)); Boucher Neuhauser syndrome NM_006702.4(PNPLA6):c.3053T>C(p.Phe1018Ser)); Palmoplantar keratoderma, mutilating, with periorificial keratotic plaques, X-linked (NM_015884.3(MBTPS2):c.1391T>C(p.Phe464Ser)); not provided (NM_001165963.1(SCN1A):c.1094T>C(p.Phe365Ser)); Deafness, autosomal recessive 7 (NM_138691.2(TMC1):c.1543T>C(p.Cys515Arg)); Congenital heart disease (NM_002052.4(GATA4):c.1147-107A>G); Ornithine carbamoyl transferase deficiency (NM_000531.5(OTC):c.238A>G(p.Lys80Glu)); not provided (NM_006245.3(PPP2R5D):c.619T>C(p.Trp207Arg)); Deficiency of UDPglucose-hexose-1-phosphateuridylyltransferase (NM_000155.3(GALT):c.499T>C(p.Trp167Arg); (m.4290T>C); Congenital muscular dystrophy, LMNA-rel (NM_170707.3(LMNA):c.1139T>C(p.Leu380Ser)); Crouzon syndrome (NM_000141.4(FGFR2):c.874A>G(p.Lys292Glu)); Homocystinuria, pyridoxine-responsive (NM_000071.2(CBS):c.1150A>G(p.Lys384Glu)); X-linked agammaglobulinemia (NM_000061.2(BTK):c.1288A>G(p.Lys430Glu)); Macular dystrophy, vitelliform, 5 (NM_016247.3(IMPG2):c.370T>C(p.Phe124Leu)); Bullous ichthyosiform erythroderma (NM_000421.3(KRT10):c.1315A>G(p.Lys439Glu)); Dominant hereditary optic atrophy (NM_015560.2(OPA1):c.1310A>G(p.Gln437Arg)); Astrocytoma; Encephalocraniocutaneous lipomatosis; Glioblastoma; Hepatocellular carcinoma; Lymphoblastic leukemia, acute, with lymphomatous features; Medulloblastoma; Transitional cell carcinoma of the bladder (NM_023110.2(FGFR1):c.1966A>G(p.Lys656Glu)); Limb-girdle muscular dystrophy, type 2L (NM_213599.2(ANO5):c.1733T>C(p.Phe578Ser)); Leber optic atrophy (m.3394T>C); not provided (NM_000531.5(OTC):c.541-2A>G); Borjeson-Forssman-Lehmann syndrome (NM_001015877.1(PHF6):c.769A>G(p.Arg257Gly)); Deficiency of UDPglucose-hexo se-1-phosphateuridylyltransferase (NM_000155.3 (GALT):c.482 T>C (p.Leu161Pro)); Atrial fibrillation, familial, 3, Atrial fibrillation (NM_000218.2(KCNQ1):c.418A>G(p.Ser140Gly)); Cardiomyopathy, mitochondrial (m.12297T>C); Cerebro-oculo-facio-skeletal syndrome (NM_000124.3(ERCC6):c.2960T>C(p.Leu987Pro)); Hereditary factor VIII deficiency disease (NM_000132.3 (F8):c.1660A>G(p.Ser554Gly)); Leukocyte adhesion deficiency (NM_000211.4(ITGB2):c.446T>C(p.Leu149Pro)); Short-rib thoracic dysplasia 3 with or without polydactyly (NM_001080463.1(DYNC2H1):c.4610A>G(p.Gln1537Arg)); Adult neuronal ceroid lipofuscinosis (NM_017882.2(CLN6):c.200T>C(p.Leu67Pro)); Mucolipidosis III Gamma (NM_032520.4(GNPTG):c.610-2A>G); Marie Unna hereditary hypotrichosis 1 (NM_005144.4(HR):c.-218A>G); Hereditary diffuse leukoencephalopathy with spheroids (NM_005211.3(CSF1R):c.2320-2A>G); Combined oxidative phosphorylation deficiency 5 (NM_020191.2(MRPS22):c.644T>C(p.Leu215Pro)); Retinitis pigmentosa, Usher syndrome, type 2A (NM_206933.2(USH2A):c.8559-2A>G); Hemophagocytic lymph histiocytosis, familial, (NM_003764.3(STX11):c.173T>C(p.Leu58Pro)); Crigler Najjar syndrome, type 1 (NM_000463.2(UGT1A1):c.1085-2A>G; NG 012088.1:g.2209A>G); Tumor predisposition syndrome (NM_004656.3(BAP1):c.438-2A>G); Spastic paraplegia 4, autosomal dominant (NM_014946.3(SPAST):c.1688-2A>G); Hereditary cancer-predisposing syndrome, Carcinoma of colon (NM_001128425.1(MUTYH):c.1187-2A>G); Familial adenomatous polyposis 1 (NM_000038.5(APC):c.1744-2A>G); Supravalvar aortic stenosis (NM_000501.3(ELN):c.890-2A>G); Combined immunodeficiency (NM_006785.3 (MALT1):c.1019-2A>G); Severe myoclonic epilepsy in infancy (NM_001165963.1(SCN1A):c.4055T>C(p.Leu1352Pro)); Medium-chain acyl-coenzyme A dehydrogenase deficiency (NM_000016.5(ACADM):c.742A>G(p.Arg248 Gly)); Mucopolysaccharidosis, MPS-II (NM_000202.7(IDS):c.1016T>C(p.Leu339Pro)); Myopathy, distal, 1 (NM_000257.3 (MYH7):c.5117T>C(p.Leu1706Pro)); beta Thalassemia (NM_000518.4(HBB):c.316-2A>G); Familial hypercholesterolemia (NM_000527.4(LDLR):c.1187-2A>G); Hereditary cancer-predisposing syndrome; Li-Fraumeni syndrome (NM_000546.5(TP53):c.1101-2A>G); Mental retardation, autosomal dominant 6 (NM_000834.3(GRIN2B):c.2360-2A>G); Succinate-semialdehyde dehydrogenase deficiency (NM_001080.3(ALDH5A1):c.610-2A>G); Deafness, autosomal recessive 2 (NM_001127180.1(MYO7A):c.4153-2A>G); Primary pulmonary hypertension (NM_001204.6(BMPR2):c.1039T>C(p.Cys347Arg)); not provided (NM_003165.3 (STXBP1):c.1060T>C(p.Cys354Arg)); Smith-Magenis Syndrome-like (NM_004187.3(KDM5C):c.3068A>G(p Lys 1023Arg)); Pachyonychia congenita 3 (NM_005554.3(KRT6A):c.1406T>C(p.Leu469Pro)); not provided (NM_005629.3(SLC6A8):c.1255-2A>G); Breast-ovarian cancer, familial 1 (NM_007294.3(BRCA1):c.4097-2A>G); Retinitis pigmentosa 80 (NM_014714.3(IFT140):c.4196T>C(p.Leu1399Pro)); Congenital generalized lipodystrophy type 2 (NM_032667.6(BSCL2):c.574-2A>G); Hyperphosphatasia with mental retardation syndrome 4 (NM_033419.4(PGAP3):c.842T>C(p.Leu281Pro)); Gaucher disease, Subacute neuronopathic (NM_000157.3(GBA):c.680A>G(p.Asn227Ser)); Homocystinuria due to CBS deficiency, Homocystinuria, pyridoxine-responsive (NM_000071.2(CBS):c.833T>C(p.Ile278Thr)); Bernard Soulier syndrome, Bernard-Souliersyndrome type C (NM_000174.4(GP9):c.70T>C(p.Cys24Arg)); Congenital disorder of glycosylation type 1J (NM_001382.3(DPAGT1):c.509A>G(p.Tyr170Cys)); Tangier disease (NM_005502.3(ABCA1):c.2804A>G(p.Asn935Ser)); Age-related macular degeneration 3 (NM_006329.3(FBLN5):c.506T>C(p.Ile169Thr)); Cohen syndrome, not specified (NM_017890.4(VPS13B):c.8978A>G(p.Asn2993Ser)); Pachyonychia congenita type 2 (NM_000422.2(KRT17):c.275A>G(p.Asn92Ser)); von Willebrand disease type 2N (NM_000552.3(VWF):c.2384A>G(p.Tyr795Cys)); Frontotemporal dementia, ubiquitin-positive (NM_002087.3(GRN):c.2T>C(p.Met1Thr)); Nephrogenic diabetes insipidus, Nephrogenicdiabetes insipidus, X-linked (NM_000054.4(AVPR2):c.614A>G(p.Tyr205Cys)); Congenital myopathy with fiber type disproportion, Central core disease (NM_000540.2(RYR1):c.1205T>C(p.Met402Thr)); Diarrhea 3, secretory sodium, congenital, syndromic (NM_021102.3(SPINT2):c.488A>G(p.Tyr163Cys)); Cardiofaciocutaneous syndrome 3, Rasopathy (NM_002755.3(MAP2K1):c.389A>G(p.Tyr130Cys); NM_002427.3(MMP13):c.272T>C(p.Met91Thr)); Familial juvenile gout (NM_003361.3(UMOD):c.383A>G(p.Asn128Ser)); Hereditary factor VIII deficiency disease (NM_000132.3(F8):c.5372T>C(p.Met1791Thr)); L-ferritin deficiency (NM_000146.3(FTL):c.1A>G(p.Met1Val)); Spermatogenic failure, X-linked, 2 (NM_001003811.1(TEX11):c.511A>G(p.Met171Val)); not provided (NM_198056.2(SCN5A):c.1247A>G(p.Tyr416Cys)); Joubert syndrome 16 (NM_016464.4(TMEM138):c.287A>G(p.His96Arg)); Cornelia de Lange syndrome 5 (NM_018486.2(HDAC8):c.1001A>G(p.His334Arg)); Severe myoclonic epilepsy in infancy (NM_001165963.1(SCN1A):c.1048A>G(p.Met350Val)); not provided (NM_000833.4(GRIN2A):c.2449A>G(p.Met817Val)); not provided (NM_000032.4(ALAS2):c.1699A>G(p.Met567Val)); Androgen resistance syndrome (NM_000044.4(AR):c.2117A>G(p.Asn706Ser)); not provided (NM_000044.4(AR):c.2659A>G(p.Met887Val)); FGFR2 related craniosynostosis (NM_000141.4(FGFR2):c.314A>G(p.Tyr105Cys)); Deficiency of UDPglucose-hexose-1-phosphate uridylyltransferase (NM_000155.3 (GALT):c.460T>C(p.Trp154Arg)); Pseudohyperkalemia Cardiff (NM_000342.3(SLC4A1):c.2201A>G(p.His734Arg)); Stargardt disease 1 (NM_000350.2(ABCA4):c.2894A>G(p.Asn965Ser)); Metachromatic leukodystrophy (NM_000487.5(ARSA):c.1274A>G(p.His425Arg)); Combined immunodeficiency; Immunodeficiency 46 (NM_003234.3(TFRC):c.58T>C(p.Tyr20His)); Inclusion body myopathy 2; Nonaka myopathy (NM_005476.5(GNE):c.1760T>C(p.Ile587Thr)); Episodic pain syndrome, familial, 3 (NM_014139.2(SCN11A):c.1142T>C(p.Ile381Thr)); Meier-Gorlin syndrome 3 (NM_014321.3(ORC6):c.2T>C(p.Met1Thr)); Inborn genetic diseases; Spastic paraplegia 4, autosomal dominant (NM_014946.3(SPAST):c.1168A>G(p.Met390Val)); not provided (NM_015335.4(MED13L):c.1A>G(p.Met1Val)); Deafness, autosomal dominant 72 (NM_025257.2(SLC44A4):c.466A>G(p.Met156Val)); Ichthyosis, congenital, autosomal recessive 13 (NM_148897.2(SDR9C7):c.599T>C(p.Ile200Thr)); Mental retardation, X-linked 61; Non-syndromic X-linked intellectual disability (NM_183353.2(RLIM):c.1067A>G(p.Tyr356Cys)); Epileptic encephalopathy, early infantile, 53 (NM_203446.2(SYNJ1):c.2663A>G(p.Tyr888Cys)); Mucopolysaccharidosis, MPS-IV-A (NM_000512.4(GALNS):c.1460A>G(p.Asn487Ser)); Multiple epiphyseal dysplasia 3 (NM_001853.3(COL9A3):c.369+2T>C); Miyoshi muscular dystrophy 1 (NM_003494.3(DYSF):c.937+2T>C); Cardio-facio-cutaneous syndrome; Rasopathy (NM_030662.3(MAP2K2):c.401A>G(p.Tyr134Cys)). An initial screen of the target motif may be performed to determine which of the engineered A3G base editors should be used for any given pathogenic SNP correction.

### IV. METHODS OF ADMINISTRATION

Any suitable cell or mammal can be administered or treated by a method or use described herein. Typically, a mammal is in need of a method described herein, that is suspected of having or expressing an abnormal or aberrant protein as a result of a C to T conversion that is associated with a disease state.

Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In certain embodiments a mammal is a human. In certain embodiments a mammal is a non-rodent mammal (e.g., human, pig, goat, sheep, horse, dog, or the like). In certain embodiments a non-rodent mammal is a human. A mammal can be any age or at any stage of development (e.g., an adult, teen, child, infant, or a mammal in utero). A mammal can be male or female. In certain embodiments a mammal can be an animal disease model, for example, animal models having or expressing an abnormal or aberrant protein that is associated with a disease state or animal models with insufficient expression of a protein, which causes a disease state.

Mammals (subjects) treated by a method or composition described herein include adults (18 years or older) and children (less than 18 years of age). Adults include the elderly. Representative adults are 50 years or older. Children range in age from 1-2 years old, or from 2-4, 4-6, 6-18, 8-10, 10-12, 12-15 and 15-18 years old. Children also include infants. Infants typically range from 1-12 months of age.

In certain embodiments, a method includes administering a plurality of viral particles or nanoparticles to a mammal as set forth herein, where severity, frequency, progression or time of onset of one or more symptoms of a disease state, decreased, reduced, prevented, inhibited or delayed. In certain embodiments, a method includes administering a plurality of viral particles or nanoparticles to a mammal to treat an adverse symptom of a disease state. In certain embodiments, a method includes administering a plurality of viral particles or nanoparticles to a mammal to stabilize, delay or prevent worsening, or progression, or reverse and adverse symptom of a disease state.

In certain embodiments a method includes administering a plurality of viral particles or nanoparticles to the patient, or portion thereof as set forth herein, of a mammal and severity, frequency, progression or time of onset of one or more symptoms of a disease state, are decreased, reduced, prevented, inhibited or delayed by at least about 5 to about 10, about 10 to about 25, about 25 to about 50, or about 50 to about 100 days.

In some embodiments, viral and non-viral based gene transfer methods can be used to introduce nucleic acids in mammalian cells or target tissues. Such methods can be used to administer nucleic acids encoding inhibitory RNAs, therapeutic proteins, or components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, 1992; Nabel & Feigner, 1993; Mitani & Caskey, 1993; Dillon, 1993; Miller, 1992; Van Brunt, 1988; Vigne, 1995; Kremer & Perricaudet, 1995; Haddada et al., 1995; and Yu et al., 1994.

Methods of non-viral delivery of nucleic acids include exosomes, lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in (e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91117424; WO 91116024. Delivery can be to cells (e.g. in vitro or ex vivo administration) or target tissues (e.g. in vivo administration).

In some embodiments, delivery is via the use of RNA or DNA viral based systems for the delivery of nucleic acids. Viral vectors in some aspects may be administered directly to patients (in vivo) or they can be used to treat cells in vitro or ex vivo, and then administered to patients. Viral-based systems in some embodiments include retroviral, lentivirus, adenoviral, adeno-associated and herpes simplex virus vectors for gene transfer.

A. Viral Vectors

The term “vector” refers to small carrier nucleic acid molecule, a plasmid, virus (e.g., AAV vector, retroviral vector, lentiviral vector), or other vehicle that can be manipulated by insertion or incorporation of a nucleic acid. Vectors, such as viral vectors, can be used to introduce/transfer nucleic acid sequences into cells, such that the nucleic acid sequence therein is transcribed and, if encoding a protein, subsequently translated by the cells.

An “expression vector” is a specialized vector that contains a gene or nucleic acid sequence with the necessary regulatory regions needed for expression in a host cell. An expression vector may contain at least an origin of replication for propagation in a cell and optionally additional elements, such as a heterologous nucleic acid sequence, expression control element (e.g., a promoter, enhancer), intron, ITR(s), and polyadenylation signal.

A viral vector is derived from or based upon one or more nucleic acid elements that comprise a viral genome. Exemplary viral vectors include adeno-associated virus (AAV) vectors, retroviral vectors, and lentiviral vectors.

The term “recombinant,” as a modifier of vector, such as recombinant viral, e.g., lenti- or parvo-virus (e.g., AAV) vectors, as well as a modifier of sequences such as recombinant nucleic acid sequences and polypeptides, means that the compositions have been manipulated (i.e., engineered) in a fashion that generally does not occur in nature. A particular example of a recombinant vector, such as an AAV, retroviral, or lentiviral vector would be where a nucleic acid sequence that is not normally present in the wild-type viral genome is inserted within the viral genome. An example of a recombinant nucleic acid sequence would be where a nucleic acid (e.g., gene) encodes an inhibitory RNA cloned into a vector, with or without 5′, 3′ and/or intron regions that the gene is normally associated within the viral genome. Although the term “recombinant” is not always used herein in reference to vectors, such as viral vectors, as well as sequences such as polynucleotides, “recombinant” forms including nucleic acid sequences, polynucleotides, transgenes, etc. are expressly included in spite of any such omission.

A recombinant viral “vector” is derived from the wild type genome of a virus, such as AAV, retrovirus, or lentivirus, by using molecular methods to remove the wild type genome from the virus, and replacing with a non-native nucleic acid, such as a nucleic acid sequence. Typically, for example, for AAV, one or both inverted terminal repeat (ITR) sequences of the AAV genome are retained in the recombinant AAV vector. A “recombinant” viral vector (e.g., rAAV) is distinguished from a viral (e.g., AAV) genome, since all or a part of the viral genome has been replaced with a non-native sequence with respect to the viral genomic nucleic acid such a nucleic acid encoding a transactivator or nucleic acid encoding an inhibitory RNA or nucleic acid encoding a therapeutic protein. Incorporation of such non-native nucleic acid sequences therefore defines the viral vector as a “recombinant” vector, which in the case of AAV can be referred to as a “rAAV vector.”

2. Adeno-Associated Virus

Adeno-associated virus (AAV) is a small nonpathogenic virus of the parvoviridae family. To date, numerous serologically distinct AAVs have been identified, and more than a dozen have been isolated from humans or primates. AAV is distinct from other members of this family by its dependence upon a helper virus for replication.

AAV genomes can exist in an extrachromosomal state without integrating into host cellular genomes; possess a broad host range; transduce both dividing and non-dividing cells in vitro and in vivo and maintain high levels of expression of the transduced genes. AAV viral particles are heat stable; resistant to solvents, detergents, changes in pH, and temperature; and can be column purified and/or concentrated on CsCl gradients or by other means. The AAV genome comprises a single-stranded deoxyribonucleic acid (ssDNA), either positive- or negative-sensed. The approximately 5 kb genome of AAV consists of one segment of single stranded DNA of either plus or minus polarity. The ends of the genome are short inverted terminal repeats (ITRs) that can fold into hairpin structures and serve as the origin of viral DNA replication.

An AAV “genome” refers to a recombinant nucleic acid sequence that is ultimately packaged or encapsulated to form an AAV particle. An AAV particle often comprises an AAV genome packaged with AAV capsid proteins. In cases where recombinant plasmids are used to construct or manufacture recombinant vectors, the AAV vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant virus production, but is not itself packaged or encapsulated into viral particles. Thus, an AAV vector “genome” refers to nucleic acid that is packaged or encapsulated by AAV capsid proteins.

The AAV virion (particle) is a non-enveloped, icosahedral particle approximately 25 nm in diameter. The AAV particle comprises an icosahedral symmetry comprised of three related capsid proteins, VP1, VP2 and VP3, which interact together to form the capsid. The right ORF often encodes the capsid proteins VP1, VP2, and VP3. These proteins are often found in a ratio of 1:1:10 respectively, but may be in varied ratios, and are all derived from the right-hand ORF. The VP1, VP2 and VP3 capsid proteins differ from each other by the use of alternative splicing and an unusual start codon. Deletion analysis has shown that removal or alteration of VP1 which is translated from an alternatively spliced message results in a reduced yield of infectious particles. Mutations within the VP3 coding region result in the failure to produce any single-stranded progeny DNA or infectious particles.

An AAV particle is a viral particle comprising an AAV capsid. In certain embodiments, the genome of an AAV particle encodes one, two or all VP1, VP2 and VP3 polypeptides.

The genome of most native AAVs often contain two open reading frames (ORFs), sometimes referred to as a left ORF and a right ORF. The left ORF often encodes the non-structural Rep proteins, Rep 40, Rep 52, Rep 68 and Rep 78, which are involved in regulation of replication and transcription in addition to the production of single-stranded progeny genomes. Two of the Rep proteins have been associated with the preferential integration of AAV genomes into a region of the q arm of human chromosome 19. Rep68/78 have been shown to possess NTP binding activity as well as DNA and RNA helicase activities. Some Rep proteins possess a nuclear localization signal as well as several potential phosphorylation sites. In certain embodiments the genome of an AAV (e.g., an rAAV) encodes some or all of the Rep proteins. In certain embodiments the genome of an AAV (e.g., an rAAV) does not encode the Rep proteins. In certain embodiments one or more of the Rep proteins can be delivered in trans and are therefore not included in an AAV particle comprising a nucleic acid encoding a polypeptide.

The ends of the AAV genome comprise short inverted terminal repeats (ITR) which have the potential to fold into T-shaped hairpin structures that serve as the origin of viral DNA replication. Accordingly, the genome of an AAV comprises one or more (e.g., a pair of) ITR sequences that flank a single stranded viral DNA genome. The ITR sequences often have a length of about 145 bases each. Within the ITR region, two elements have been described which are believed to be central to the function of the ITR, a GAGC repeat motif and the terminal resolution site (trs). The repeat motif has been shown to bind Rep when the ITR is in either a linear or hairpin conformation. This binding is thought to position Rep68/78 for cleavage at the trs which occurs in a site- and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

In certain embodiments, an AAV (e.g., a rAAV) comprises two ITRs. In certain embodiments, an AAV (e.g., a rAAV) comprises a pair of ITRs. In certain embodiments, an AAV (e.g., a rAAV) comprises a pair of ITRs that flank (i.e., are at each 5′ and 3′ end) of a nucleic acid sequence that at least encodes a polypeptide having function or activity.

An AAV vector (e.g., rAAV vector) can be packaged and is referred to herein as an “AAV particle” for subsequent infection (transduction) of a cell, ex vivo, in vitro or in vivo. Where a recombinant AAV vector is encapsulated or packaged into an AAV particle, the particle can also be referred to as a “rAAV particle.” In certain embodiments, an AAV particle is a rAAV particle. A rAAV particle often comprises a rAAV vector, or a portion thereof. A rAAV particle can be one or more rAAV particles (e.g., a plurality of AAV particles). rAAV particles typically comprise proteins that encapsulate or package the rAAV vector genome (e.g., capsid proteins). It is noted that reference to a rAAV vector can also be used to reference a rAAV particle.

Any suitable AAV particle (e.g., rAAV particle) can be used for a method or use herein. A rAAV particle, and/or genome comprised therein, can be derived from any suitable serotype or strain of AAV. A rAAV particle, and/or genome comprised therein, can be derived from two or more serotypes or strains of AAV. Accordingly, a rAAV can comprise proteins and/or nucleic acids, or portions thereof, of any serotype or strain of AAV, wherein the AAV particle is suitable for infection and/or transduction of a mammalian cell. Non-limiting examples of AAV serotypes include AAV1, AAV2, AAV3, AAV4, AAV5, AAV6, AAV7, AAV8, AAV9, AAV10, AAV11, AAV12, AAV-rh74, AAV-rh10 and AAV-2i8.

In certain embodiments a plurality of rAAV particles comprises particles of, or derived from, the same strain or serotype (or subgroup or variant). In certain embodiments a plurality of rAAV particles comprise a mixture of two or more different rAAV particles (e.g., of different serotypes and/or strains).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

In certain embodiments, a rAAV particle excludes certain serotypes. In one embodiment, a rAAV particle is not an AAV4 particle. In certain embodiments, a rAAV particle is antigenically or immunologically distinct from AAV4. Distinctness can be determined by standard methods. For example, ELISA and Western blots can be used to determine whether a viral particle is antigenically or immunologically distinct from AAV4. Furthermore, in certain embodiments a rAAV2 particle retains tissue tropism distinct from AAV4.

In certain embodiments, a rAAV vector based upon a first serotype genome corresponds to the serotype of one or more of the capsid proteins that package the vector. For example, the serotype of one or more AAV nucleic acids (e.g., ITRs) that comprises the AAV vector genome corresponds to the serotype of a capsid that comprises the rAAV particle.

In certain embodiments, a rAAV vector genome can be based upon an AAV (e.g., AAV2) serotype genome distinct from the serotype of one or more of the AAV capsid proteins that package the vector. For example, a rAAV vector genome can comprise AAV2 derived nucleic acids (e.g., ITRs), whereas at least one or more of the three capsid proteins are derived from a different serotype, e.g., an AAV1, AAV3, AAV4, AAV5, AAV6, AAV7, AAV8, AAV9, AAV10, AAV11, AAV12, Rh10, Rh74 or AAV-2i8 serotype or variant thereof.

In certain embodiments, a rAAV particle or a vector genome thereof related to a reference serotype has a polynucleotide, polypeptide or subsequence thereof that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a polynucleotide, polypeptide or subsequence of an AAV1, AAV2, AAV3, AAV4, AAV5, AAV6, AAV7, AAV8, AAV9, AAV10, AAV11, AAV12, Rh10, Rh74 or AAV-2i8 particle. In particular embodiments, a rAAV particle or a vector genome thereof related to a reference serotype has a capsid or ITR sequence that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a capsid or ITR sequence of an AAV1, AAV2, AAV3, AAV4, AAV5, AAV6, AAV7, AAV8, AAV9, AAV10, AAV11, AAV12, Rh10, Rh74 or AAV-2i8 serotype.

In certain embodiments, a method herein comprises use, administration or delivery of a rAAV1, rAAV2, rAAV3, rAAV4, rAAV5, rAAV6, rAAV7, rAAV8, rAAV9, rAAV10, rAAV11, rAAV12, rRh10, rRh74 or rAAV-2i8 particle.

In certain embodiments, a method herein comprises use, administration or delivery of a rAAV2 particle. In certain embodiments a rAAV2 particle comprises an AAV2 capsid. In certain embodiments a rAAV2 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments a rAAV2 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments, a rAAV2 particle is a variant of a native or wild-type AAV2 particle. In some aspects, one or more capsid proteins of an AAV2 variant have 1, 2, 3, 4, 5, 5-10, 10-15, 15-20 or more amino acid substitutions compared to capsid protein(s) of a native or wild-type AAV2 particle.

In certain embodiments a rAAV9 particle comprises an AAV9 capsid. In certain embodiments a rAAV9 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments a rAAV9 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments, a rAAV9 particle is a variant of a native or wild-type AAV9 particle. In some aspects, one or more capsid proteins of an AAV9 variant have 1, 2, 3, 4, 5, 5-10, 10-15, 15-20 or more amino acid substitutions compared to capsid protein(s) of a native or wild-type AAV9 particle.

In certain embodiments, a rAAV particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV1, AAV2, AAV3, AAV4, AAV5, AAV6, AAV7, AAV8, AAV9, AAV10, AAV11, AAV12, AAV-rh74, AAV-rh10 or AAV-2i8, as long as they retain one or more desired ITR functions (e.g., ability to form a hairpin, which allows DNA replication; integration of the AAV DNA into a host cell genome; and/or packaging, if desired).

In certain embodiments, a rAAV2 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

In certain embodiments, a rAAV9 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

A rAAV particle can comprise an ITR having any suitable number of “GAGC” repeats. In certain embodiments an ITR of an AAV2 particle comprises 1, 2, 3, 4, 5, 6, 7, 8, 9 or 10 or more “GAGC” repeats. In certain embodiments a rAAV2 particle comprises an ITR comprising three “GAGC” repeats. In certain embodiments a rAAV2 particle comprises an ITR which has less than four “GAGC” repeats. In certain embodiments a rAAV2 particle comprises an ITR which has more than four “GAGC” repeats. In certain embodiments an ITR of a rAAV2 particle comprises a Rep binding site wherein the fourth nucleotide in the first two “GAGC” repeats is a C rather than a T.

Exemplary suitable length of DNA can be incorporated in rAAV vectors for packaging/encapsidation into a rAAV particle can about 5 kilobases (kb) or less. In particular, embodiments, length of DNA is less than about 5 kb, less than about 4.5 kb, less than about 4 kb, less than about 3.5 kb, less than about 3 kb, or less than about 2.5 kb.

rAAV vectors that include a nucleic acid sequence that directs the expression of an RNAi or polypeptide can be generated using suitable recombinant techniques known in the art (e.g., see Sambrook et al., 1989). Recombinant AAV vectors are typically packaged into transduction-competent AAV particles and propagated using an AAV viral packaging system. A transduction-competent AAV particle is capable of binding to and entering a mammalian cell and subsequently delivering a nucleic acid cargo (e.g., a heterologous gene) to the nucleus of the cell. Thus, an intact rAAV particle that is transduction-competent is configured to transduce a mammalian cell. A rAAV particle configured to transduce a mammalian cell is often not replication competent, and requires additional protein machinery to self-replicate. Thus, a rAAV particle that is configured to transduce a mammalian cell is engineered to bind and enter a mammalian cell and deliver a nucleic acid to the cell, wherein the nucleic acid for delivery is often positioned between a pair of AAV ITRs in the rAAV genome.

Suitable host cells for producing transduction-competent AAV particles include but are not limited to microorganisms, yeast cells, insect cells, and mammalian cells that can be, or have been, used as recipients of a heterologous rAAV vectors. Cells from the stable human cell line, HEK293 (readily available through, e.g., the American Type Culture Collection under Accession Number ATCC CRL1573) can be used. In certain embodiments a modified human embryonic kidney cell line (e.g., HEK293), which is transformed with adenovirus type-5 DNA fragments, and expresses the adenoviral Ela and E1b genes is used to generate recombinant AAV particles. The modified HEK293 cell line is readily transfected, and provides a particularly convenient platform in which to produce rAAV particles. Methods of generating high titer AAV particles capable of transducing mammalian cells are known in the art. For example, AAV particle can be made as set forth in Wright, 2008 and Wright, 2009.

In certain embodiments, AAV helper functions are introduced into the host cell by transfecting the host cell with an AAV helper construct either prior to, or concurrently with, the transfection of an AAV expression vector. AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

3. Retrovirus

Viral vectors for use as a delivered agent in the methods, compositions and uses herein include a retroviral vector (see e.g., Miller (1992) Nature, 357:455-460). Retroviral vectors are well suited for delivering nucleic acid into cells because of their ability to deliver an unrearranged, single copy gene into a broad range of rodent, primate and human somatic cells. Retroviral vectors integrate into the genome of host cells. Unlike other viral vectors, they only infect dividing cells.

Retroviruses are RNA viruses such that the viral genome is RNA. When a host cell is infected with a retrovirus, the genomic RNA is reverse transcribed into a DNA intermediate, which is integrated very efficiently into the chromosomal DNA of infected cells. This integrated DNA intermediate is referred to as a provirus. Transcription of the provirus and assembly into infectious virus occurs in the presence of an appropriate helper virus or in a cell line containing appropriate sequences permitting encapsulation without coincident production of a contaminating helper virus. A helper virus is not required for the production of the recombinant retrovirus if the sequences for encapsulation are provided by co-transfection with appropriate vectors.

The retroviral genome and the proviral DNA have three genes: the gag, the pol and the env, which are flanked by two long terminal repeat (LTR) sequences. The gag gene encodes the internal structural (matrix, capsid, and nucleocapsid) proteins and the env gene encodes viral envelope glycoproteins. The pol gene encodes products that include the RNA-directed DNA polymerase reverse transcriptase that transcribes the viral RNA into double-stranded DNA, integrase that integrate the DNA produced by reverse transcriptase into host chromosomal DNA, and protease that acts to process the encoded gag and pol genes. The 5′ and 3′ LTRs serve to promote transcription and polyadenylation of the virion RNAs. The LTR contains all other cis-acting sequences necessary for viral replication.

Retroviral vectors are described by Coffin et al., Retroviruses, Cold Spring Harbor Laboratory Press (1997). Exemplary of a retrovirus is Moloney murine leukemia virus (MMLV) or the murine stem cell virus (MSCV). Retroviral vectors can be replication-competent or replication-defective. Typically, a retroviral vector is replication-defective in which the coding regions for genes necessary for additional rounds of virion replication and packaging are deleted or replaced with other genes. Consequently, the viruses are not able to continue their typical lytic pathway once an initial target cell is infected. Such retroviral vectors, and the necessary agents to produce such viruses (e.g., packaging cell line) are commercially available (see, e.g., retroviral vectors and systems available from Clontech, such as Catalog number 634401, 631503, 631501, and others, Clontech, Mountain View, Calif.).

Such retroviral vectors can be produced as delivered agents by replacing the viral genes required for replication with the nucleic acid molecule to be delivered. The resulting genome contains an LTR at each end with the desired gene or genes in between. Methods of producing retrovirus are known to one of skill in the art (see, e.g., International published PCT Application No. WO1995/026411). The retroviral vector can be produced in a packaging cell line containing a helper plasmid or plasmids. The packaging cell line provides the viral proteins required for capsid production and the virion maturation of the vector (e.g., gag, pol and env genes). Typically, at least two separate helper plasmids (separately containing the gag and pol genes; and the env gene) are used so that recombination between the vector plasmid cannot occur. For example, the retroviral vector can be transferred into a packaging cell line using standard methods of transfection, such as calcium phosphate mediated transfection. Packaging cell lines are well known to one of skill in the art, and are commercially available. An exemplary packaging cell line is GP2-293 packaging cell line (Catalog Numbers 631505, 631507, 631512, Clontech). After sufficient time for virion product, the virus is harvested. If desired, the harvested virus can be used to infect a second packaging cell line, for example, to produce a virus with varied host tropism. The end result is a replicative incompetent recombinant retrovirus that includes the nucleic acid of interest but lacks the other structural genes such that a new virus cannot be formed in the host cell.

References illustrating the use of retroviral vectors in gene therapy include: Clowes et al., (1994) J. Clin. Invest. 93:644-651; Kiem et al., (1994) Blood 83:1467-1473; Salmons and Gunzberg (1993) Human Gene Therapy 4:129-141; Grossman and Wilson (1993) Curr. Opin. in Genetics and Devel. 3:110-114; Sheridan (2011) Nature Biotechnology, 29:121; Cassani et al. (2009) Blood, 114:3546-3556.

4. Lentivirus

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe. Lentiviral vectors are well known in the art (see, e.g., U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell, wherein a suitable host cell is transfected with two or more vectors carrying the packaging functions, namely gag, pol and env, as well as rev and tat, is described in U.S. Pat. No. 5,994,136, incorporated herein by reference.

The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTRs serve to promote transcription and polyadenylation of the virion RNAs. The LTR contains all other cis-acting sequences necessary for viral replication. Lentiviruses have additional genes including vif, vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

5. Other Viral Vectors

The development and utility of viral vectors for gene delivery is constantly improving and evolving. Other viral vectors such as poxvirus; e.g., vaccinia virus (Gnant et al., 1999; Gnant et al., 1999), alpha virus; e.g., sindbis virus, Semliki forest virus (Lundstrom, 1999), reovirus (Coffey et al., 1998) and influenza A virus (Neumann et al., 1999) are contemplated for use in the present disclosure and may be selected according to the requisite properties of the target system.

6. Chimeric Viral Vectors

Chimeric or hybrid viral vectors are being developed for use in therapeutic gene delivery and are contemplated for use in the present disclosure. Chimeric poxviral/retroviral vectors (Holzer et al., 1999), adenoviral/retroviral vectors (Feng et al., 1997; Bilbao et al., 1997; Caplen et al., 2000) and adenoviral/adeno-associated viral vectors (Fisher et al., 1996; U.S. Pat. No. 5,871,982) have been described. These “chimeric” viral gene transfer systems can exploit the favorable features of two or more parent viral species. For example, Wilson et al., provide a chimeric vector construct which comprises a portion of an adenovirus, AAV 5′ and 3′ ITR sequences and a selected transgene, described below (U.S. Pat. No. 5,871,983, specifically incorporate herein by reference).

B. Nanoparticles

1. Lipid-Based Nanoparticles

In some embodiments, a lipid-based nanoparticle is a liposome, an exosome, a lipid preparation, or another lipid-based nanoparticle, such as a lipid-based vesicle (e.g., a DOTAP:cholesterol vesicle). Lipid-based nanoparticles may be positively charged, negatively charged, or neutral.

b. Liposomes

A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. Liposomes provided herein include unilamellar liposomes, multilamellar liposomes, and multivesicular liposomes. Liposomes provided herein may be positively charged, negatively charged, or neutrally charged. In certain embodiments, the liposomes are neutral in charge.

A multilamellar liposome has multiple lipid layers separated by aqueous medium. Such liposomes form spontaneously when lipids comprising phospholipids are suspended in an excess of aqueous solution. The lipid components undergo self-rearrangement before the formation of closed structures and entrap water and dissolved solutes between the lipid bilayers. Lipophilic molecules or molecules with lipophilic regions may also dissolve in or associate with the lipid bilayer.

In specific aspects, a polypeptide, a nucleic acid, or a small molecule drug may be, for example, encapsulated in the aqueous interior of a liposome, interspersed within the lipid bilayer of a liposome, attached to a liposome via a linking molecule that is associated with both the liposome and the polypeptide/nucleic acid, entrapped in a liposome, complexed with a liposome, or the like.

A liposome used according to the present embodiments can be made by different methods, as would be known to one of ordinary skill in the art. For example, a phospholipid, such as for example the neutral phospholipid dioleoylphosphatidylcholine (DOPC), is dissolved in tert-butanol. The lipid(s) is then mixed with a polypeptide, nucleic acid, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min to 2 h, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

The dried lipids or lyophilized liposomes prepared as described above may be dehydrated and reconstituted in a solution of a protein or peptide and diluted to an appropriate concentration with a suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000×g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

Additional liposomes which may be useful with the present embodiments include cationic liposomes, for example, as described in WO02/100435A1, U.S. Pat. No. 5,962,016, U.S. Application 2004/0208921, WO03/015757A1, WO04029213A2, U.S. Pat. Nos. 5,030,453, and 6,680,068, all of which are hereby incorporated by reference in their entirety without disclaimer.

In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040, each incorporated herein by reference.

In certain embodiments, the lipid-based nanoparticle is a neutral liposome (e.g., a DOPC liposome). “Neutral liposomes” or “non-charged liposomes”, as used herein, are defined as liposomes having one or more lipid components that yield an essentially-neutral, net charge (substantially non-charged). By “essentially neutral” or “essentially non-charged”, it is meant that few, if any, lipid components within a given population (e.g., a population of liposomes) include a charge that is not canceled by an opposite charge of another component (i.e., fewer than 10% of components include a non-canceled charge, more preferably fewer than 5%, and most preferably fewer than 1%). In certain embodiments, neutral liposomes may include mostly lipids and/or phospholipids that are themselves neutral under physiological conditions (i.e., at about pH 7).

Liposomes and/or lipid-based nanoparticles of the present embodiments may comprise a phospholipid. In certain embodiments, a single kind of phospholipid may be used in the creation of liposomes (e.g., a neutral phospholipid, such as DOPC, may be used to generate neutral liposomes). In other embodiments, more than one kind of phospholipid may be used to create liposomes. Phospholipids may be from natural or synthetic sources. Phospholipids include, for example, phosphatidylcholines, phosphatidylglycerols, and phosphatidylethanolamines; because phosphatidylethanolamines and phosphatidyl cholines are non-charged under physiological conditions (i.e., at about pH 7), these compounds may be particularly useful for generating neutral liposomes. In certain embodiments, the phospholipid DOPC is used to produce non-charged liposomes. In certain embodiments, a lipid that is not a phospholipid (e.g., a cholesterol) may be used

Phospholipids include glycerophospholipids and certain sphingolipids. Phospholipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), distearoyl sphingomyelin (“DSSP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine.

c. Exosomes

“Extracellular vesicles” and “EVs” are cell-derived and cell-secreted microvesicles which, as a class, include exosomes, exosome-like vesicles, ectosomes (which result from budding of vesicles directly from the plasma membrane), microparticles, microvesicles, shedding microvesicles (SMVs), nanoparticles and even (large) apoptotic blebs or bodies (resulting from cell death) or membrane particles.

The terms “microvesicle” and “exosomes,” as used herein, refer to a membranous particle having a diameter (or largest dimension where the particles is not spheroid) of between about 10 nm to about 5000 nm, more typically between 30 nm and 1000 nm, and most typically between about 50 nm and 750 nm, wherein at least part of the membrane of the exosomes is directly obtained from a cell. Most commonly, exosomes will have a size (average diameter) that is up to 5% of the size of the donor cell. Therefore, especially contemplated exosomes include those that are shed from a cell.

Exosomes may be detected in or isolated from any suitable sample type, such as, for example, body fluids. As used herein, the term “isolated” refers to separation out of its natural environment and is meant to include at least partial purification and may include substantial purification. As used herein, the term “sample” refers to any sample suitable for the methods provided by the present invention. The sample may be any sample that includes exosomes suitable for detection or isolation. Sources of samples include blood, bone marrow, pleural fluid, peritoneal fluid, cerebrospinal fluid, urine, saliva, amniotic fluid, malignant ascites, broncho-alveolar lavage fluid, synovial fluid, breast milk, sweat, tears, joint fluid, and bronchial washes. In one aspect, the sample is a blood sample, including, for example, whole blood or any fraction or component thereof. A blood sample suitable for use with the present invention may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer.

Exosomes may also be isolated from tissue samples, such as surgical samples, biopsy samples, tissues, feces, and cultured cells. When isolating exosomes from tissue sources it may be necessary to homogenize the tissue in order to obtain a single cell suspension followed by lysis of the cells to release the exosomes. When isolating exosomes from tissue samples it is important to select homogenization and lysis procedures that do not result in disruption of the exosomes. Exosomes contemplated herein are preferably isolated from body fluid in a physiologically acceptable solution, for example, buffered saline, growth medium, various aqueous medium, etc.

Exosomes may be isolated from freshly collected samples or from samples that have been stored frozen or refrigerated. In some embodiments, exosomes may be isolated from cell culture medium. Although not necessary, higher purity exosomes may be obtained if fluid samples are clarified before precipitation with a volume-excluding polymer, to remove any debris from the sample. Methods of clarification include centrifugation, ultracentrifugation, filtration, or ultrafiltration. Most typically, exosomes can be isolated by numerous methods well-known in the art. One preferred method is differential centrifugation from body fluids or cell culture supernatants. Exemplary methods for isolation of exosomes are described in (Losche et al., 2004; Mesri and Altieri, 1998; Morel et al., 2004). Alternatively, exosomes may also be isolated via flow cytometry as described in (Combes et al., 1997).

One accepted protocol for isolation of exosomes includes ultracentrifugation, often in combination with sucrose density gradients or sucrose cushions to float the relatively low-density exosomes. Isolation of exosomes by sequential differential centrifugations is complicated by the possibility of overlapping size distributions with other microvesicles or macromolecular complexes. Furthermore, centrifugation may provide insufficient means to separate vesicles based on their sizes. However, sequential centrifugations, when combined with sucrose gradient ultracentrifugation, can provide high enrichment of exosomes.

Isolation of exosomes based on size, using alternatives to the ultracentrifugation routes, is another option. Successful purification of exosomes using ultrafiltration procedures that are less time consuming than ultracentrifugation, and do not require use of special equipment have been reported. Similarly, a commercial kit is available (EXOMIR™, Bioo Scientific) which allows removal of cells, platelets, and cellular debris on one microfilter and capturing of vesicles bigger than 30 nm on a second microfilter using positive pressure to drive the fluid. However, for this process, the exosomes are not recovered, their RNA content is directly extracted from the material caught on the second microfilter, which can then be used for PCR analysis. HPLC-based protocols could potentially allow one to obtain highly pure exosomes, though these processes require dedicated equipment and are difficult to scale up. A significant problem is that both blood and cell culture media contain large numbers of nanoparticles (some non-vesicular) in the same size range as exosomes. For example, some miRNAs may be contained within extracellular protein complexes rather than exosomes; however, treatment with protease (e.g., proteinase K) can be performed to eliminate any possible contamination with “extraexosomal” protein.

In another embodiment, exosomes may be captured by techniques commonly used to enrich a sample for exosomes, such as those involving immunospecific interactions (e.g., immunomagnetic capture). Immunomagnetic capture, also known as immunomagnetic cell separation, typically involves attaching antibodies directed to proteins found on a particular cell type to small paramagnetic beads. When the antibody-coated beads are mixed with a sample, such as blood, they attach to and surround the particular cell. The sample is then placed in a strong magnetic field, causing the beads to pellet to one side. After removing the blood, captured cells are retained with the beads. Many variations of this general method are well-known in the art and suitable for use to isolate exosomes. In one example, the exosomes may be attached to magnetic beads (e.g., aldehyde/sulphate beads) and then an antibody is added to the mixture to recognize an epitope on the surface of the exosomes that are attached to the beads.

As will be appreciated by one of skill in the art, prior or subsequent to loading with cargo, exosomes may be further altered by inclusion of a targeting moiety to enhance the utility thereof as a vehicle for delivery of cargo. In this regard, exosomes may be engineered to incorporate an entity that specifically targets a particular cell to tissue type. This target-specific entity, e.g., peptide having affinity for a receptor or ligand on the target cell or tissue, may be integrated within the exosomal membrane, for example, by fusion to an exosomal membrane marker using methods well-established in the art.

2. Nonlipid Nanoparticles

Spherical Nucleic Acid (SNA™) constructs and other nanoparticles (particularly gold nanoparticles) are also contemplated as a means to deliver chimeric minigenes to intended target cells. Due to their dense loading, a majority of cargo (e.g., DNA) remains bound to the constructs inside cells, conferring nucleic acid stability and resistance to enzymatic degradation. For all cell types studied (e.g., neurons, tumor cell lines, etc.) the constructs demonstrate a transfection efficiency of 99% with no need for carriers or transfection agents. The unique target binding affinity and specificity of the constructs allow exquisite specificity for matched target sequences (i.e., limited off-target effects). The constructs significantly outperform leading conventional transfection reagents (Lipofectamine 2000 and Cytofectin). The constructs can enter a variety of cultured cells, primary cells, and tissues with no apparent toxicity. The constructs elicit minimal changes in global gene expression as measured by whole-genome microarray studies and cytokine-specific protein assays. Any number of single or combinatorial agents (e.g., proteins, peptides, small molecules) can be used to tailor the surface of the constructs. See, e.g., Jensen et al., Sci. Transl. Med. 5, 209ra152 (2013).

Self-assembling nanoparticles with nucleic acid cargo may be constructed with polyethyleneimine (PEI) that is PEGylated with an Arg-Gly-Asp (RGD) peptide ligand attached at the distal end of the polyethylene glycol (PEG). Nanoplexes may be prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in the formation of polyplexes with average particle size distribution of about 100 nm, hence referred to here as nanoplexes (see, e.g., Bartlett et al., PNAS, 104:39, 2007).

### V. PHARMACEUTICAL COMPOSITIONS

As used herein the term “pharmaceutically acceptable” and “physiologically acceptable” mean a biologically acceptable composition, formulation, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Such composition, “pharmaceutically acceptable” and “physiologically acceptable” formulations and compositions can be sterile. Such pharmaceutical formulations and compositions may be used, for example in administering a viral particle or nanoparticle to a subject.

Such formulations and compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Supplementary active compounds (e.g., preservatives, antibacterial, antiviral and antifungal agents) can also be incorporated into the formulations and compositions.

Pharmaceutical compositions typically contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as surfactants, wetting or emulsifying agents, pH buffering substances, and the like, may be present in such vehicles.

Pharmaceutical compositions can be formulated to be compatible with a particular route of administration or delivery, as set forth herein or known to one of skill in the art. Thus, pharmaceutical compositions include carriers, diluents, or excipients suitable for administration or delivery by various routes.

Pharmaceutical forms suitable for injection or infusion of viral particles or nanoparticles can include sterile aqueous solutions or dispersions which are adapted for the extemporaneous preparation of sterile injectable or infusible solutions or dispersions, optionally encapsulated in liposomes. In all cases, the ultimate form should be a sterile fluid and stable under the conditions of manufacture, use and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Isotonic agents, for example, sugars, buffers or salts (e.g., sodium chloride) can be included. Prolonged absorption of injectable compositions can be brought about by the use in the compositions of agents delaying absorption, for example, aluminum monostearate and gelatin.

Solutions or suspensions of viral particles or nanoparticles can optionally include one or more of the following components: a sterile diluent such as water for injection, saline solution, such as phosphate buffered saline (PBS), artificial CSF, a surfactants, fixed oils, a polyol (for example, glycerol, propylene glycol, and liquid polyethylene glycol, and the like), glycerin, or other synthetic solvents; antibacterial and antifungal agents such as parabens, chlorobutanol, phenol, ascorbic acid, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Pharmaceutical formulations, compositions and delivery systems appropriate for the compositions, methods and uses of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

Viral particles, nanoparticles, and their compositions may be formulated in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms are dependent upon the number of viral particles or nanoparticles believed necessary to produce the desired effect(s). The amount necessary can be formulated in a single dose, or can be formulated in multiple dosage units. The dose may be adjusted to a suitable viral particle or nanoparticle concentration, optionally combined with an anti-inflammatory agent, and packaged for use.

In one embodiment, pharmaceutical compositions will include sufficient genetic material to provide a therapeutically effective amount, i.e., an amount sufficient to reduce or ameliorate symptoms or an adverse effect of a disease state in question or an amount sufficient to confer the desired benefit.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Thus, for example, viral particles, nanoparticles, and pharmaceutical compositions thereof can be packaged in single or multiple unit dosage form for ease of administration and uniformity of dosage.

Formulations containing viral particles or nanoparticles typically contain an effective amount, the effective amount being readily determined by one skilled in the art. The viral particles or nanoparticles may typically range from about 1% to about 95% (w/w) of the composition, or even higher if suitable. The quantity to be administered depends upon factors such as the age, weight and physical condition of the mammal or the human subject considered for treatment. Effective dosages can be established by one of ordinary skill in the art through routine trials establishing dose response curves.

### VI. DEFINITIONS

The terms “polynucleotide,” “nucleic acid” and “transgene” are used interchangeably herein to refer to all forms of nucleic acid, oligonucleotides, including deoxyribonucleic acid (DNA) and ribonucleic acid (RNA) and polymers thereof. Polynucleotides include genomic DNA, cDNA and antisense DNA, and spliced or unspliced mRNA, rRNA, tRNA and inhibitory DNA or RNA (RNAi, e.g., small or short hairpin (sh)RNA, microRNA (miRNA), small or short interfering (si)RNA, trans-splicing RNA, or antisense RNA). Polynucleotides can include naturally occurring, synthetic, and intentionally modified or altered polynucleotides (e.g., variant nucleic acid). Polynucleotides can be single stranded, double stranded, or triplex, linear or circular, and can be of any suitable length. In discussing polynucleotides, a sequence or structure of a particular polynucleotide may be described herein according to the convention of providing the sequence in the 5′ to 3′ direction.

A nucleic acid encoding a polypeptide often comprises an open reading frame that encodes the polypeptide. Unless otherwise indicated, a particular nucleic acid sequence also includes degenerate codon substitutions.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism.

A “promoter” refers to a nucleotide sequence, usually upstream (5′) of a coding sequence, which directs and/or controls the expression of the coding sequence by providing the recognition for RNA polymerase and other factors required for proper transcription. “Promoter” includes a minimal promoter that is a short DNA sequence comprised of a TATA-box and optionally other sequences that serve to specify the site of transcription initiation, to which regulatory elements are added for control of expression.

An “enhancer” is a DNA sequence that can stimulate transcription activity and may be an innate element of the promoter or a heterologous element that enhances the level or tissue specificity of expression. It is capable of operating in either orientation (5′->3′ or 3′->5′), and may be capable of functioning even when positioned either upstream or downstream of the promoter.

Promoters and/or enhancers may be derived in their entirety from a native gene, or be composed of different elements derived from different elements found in nature, or even be comprised of synthetic DNA segments. A promoter or enhancer may comprise DNA sequences that are involved in the binding of protein factors that modulate/control effectiveness of transcription initiation in response to stimuli, physiological or developmental conditions.

Non-limiting examples include SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

A “transgene” is used herein to conveniently refer to a nucleic acid sequence/polynucleotide that is intended or has been introduced into a cell or organism. Transgenes include any nucleic acid, such as a gene that encodes an inhibitory RNA or polypeptide or protein, and are generally heterologous with respect to naturally occurring AAV genomic sequences.

The term “transduce” refers to introduction of a nucleic acid sequence into a cell or host organism by way of a vector (e.g., a viral particle). Introduction of a transgene into a cell by a viral particle is can therefore be referred to as “transduction” of the cell. The transgene may or may not be integrated into genomic nucleic acid of a transduced cell. If an introduced transgene becomes integrated into the nucleic acid (genomic DNA) of the recipient cell or organism it can be stably maintained in that cell or organism and further passed on to or inherited by progeny cells or organisms of the recipient cell or organism. Finally, the introduced transgene may exist in the recipient cell or host organism extra chromosomally, or only transiently. A “transduced cell” is therefore a cell into which the transgene has been introduced by way of transduction. Thus, a “transduced” cell is a cell into which, or a progeny thereof in which a transgene has been introduced. A transduced cell can be propagated, transgene transcribed and the encoded inhibitory RNA or protein expressed. For gene therapy uses and methods, a transduced cell can be in a mammal.

A nucleic acid/transgene is “operably linked” when it is placed into a functional relationship with another nucleic acid sequence. A nucleic acid/transgene encoding and RNAi or a polypeptide, or a nucleic acid directing expression of a polypeptide may include an inducible promoter, or a tissue-specific promoter for controlling transcription of the encoded polypeptide. A nucleic acid operably linked to an expression control element can also be referred to as an expression cassette.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation, e.g., a missense or nonsense mutation.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a protein encoded thereby, and some codons of the nucleic acid variant will be changed which in turn changes the amino acids of a protein encoded thereby.

The terms “protein” and “polypeptide” are used interchangeably herein. The “polypeptides” encoded by a “nucleic acid” or “polynucleotide” or “transgene” disclosed herein include partial or full-length native sequences, as with naturally occurring wild-type and functional polymorphic proteins, functional subsequences (fragments) thereof, and sequence variants thereof, so long as the polypeptide retains some degree of function or activity. Accordingly, in methods and uses of the invention, such polypeptides encoded by nucleic acid sequences are not required to be identical to the endogenous protein that is defective, or whose activity, function, or expression is insufficient, deficient or absent in a treated mammal.

An example of an amino acid modification is a conservative amino acid substitution or a deletion. In particular embodiments, a modified or variant sequence retains at least part of a function or activity of the unmodified sequence (e.g., wild-type sequence).

Another example of an amino acid modification is a targeting peptide introduced into a capsid protein of a viral particle. Peptides have been identified that target recombinant viral vectors or nanoparticles to various organs and tissues.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

“Conservative variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are identical is that one polypeptide is immunologically reactive with antibodies raised against the second polypeptide. Thus, a polypeptide is identical to a second polypeptide, for example, where the two peptides differ only by a conservative substitution.

The terms “treat” and “treatment” refer to both therapeutic treatment and prophylactic or preventative measures, wherein the object is to prevent, inhibit, reduce, or decrease an undesired physiological change or disorder, such as the development, progression or worsening of the disorder. For purposes of this invention, beneficial or desired clinical results include, but are not limited to, alleviation of symptoms, diminishment of extent of disease, stabilizing a (i.e., not worsening or progressing) symptom or adverse effect of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those predisposed (e.g., as determined by a genetic assay).

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1 Materials and Methods

General cell culture. HEK293T cells (ATCC CRL-3216) were cultured using DMEM (ThermoFisher Scientific #10569044) supplemented with 10% fetal bovine serum (ThermoFisher Scientific #10437028) and 1× penicillin-streptomycin (ThermoFisher Scientific #15140122) in T-75 flasks (Corning 353136). Cells were passaged every two days at 90-95% confluency. Cells were washed with PBS (ThermoFisher Scientific #10010023), detached using Tryple Express (ThermoFisher Scientific #12605010), and spun down in centrifuge at 200×g for 3 min. The supplemented DMEM was used to resuspend cells and a quarter of the volume was used to culture into a new flask. For transfection, 35k-45k cells were plated into each well of Poly-D-Lysine coated 48-well plates (Corning 356509). Cells were incubated at 37° C. for 16 h before transfection.

Transfection of HEK293T cells. In a PCR tube, 750 ng of base editor plasmid and 250 ng of sgRNA plasmid were mixed and diluted up to 12.5 μL using Opti-MEM (ThermoFisher Scientific #31985062). In a separate tube, 1.5 μL of lipofectamine 2000 (ThermoFisher Scientific #11668027) and 11.0 μL of Opti-MEM were mixed, and then transferred into the tube containing the plasmid mix. After a 15 min incubation at room temperature, the plasmid-lipofectamine complex was added on top of a 48-well culture with cells dropwise. Cells were incubated at 37° C. for 60-72 h without changing media.

Base editing analysis using EditR. At 3-days post-transfection, cell media was aspirated, washed with 100 μL PBS per well, and incubated with lysis buffer (10 mM Tris-HCl, 0.05% SDS, 25 μg/mL proteinase K, pH 8.0) at 37° C. for 1 h. Cell lysates were collected in PCR tubes and incubated at 80° C. for 30 min to inactivate proteinase K. Regions of interest of genomic DNA were amplified using Phanta Max (Vazyme #P515) and 29-34 PCR cycles. Amplified fragments were gel extracted and sent for Sanger sequencing using Genewiz. Sanger chromatograms were analyzed using EditR (available at moriaritylab.shinyapps.io/editr_v10) to quantify the percentage of C-to-T conversion at the target region.

Creating stable cell line disease model. The HEK293T stable cell line was constructed by cloning a 200-bp fragment of disease-associated gene upstream of an EF1α promoter to drive the expression of the puromycin resistance gene in a lentiviral vector. The single-base mutation of a disease-associated gene was inserted by PCR and In-Fusion cloning (Takara). The lentiviral vector was transfected into HEK293T cells in a 24-well plate (Olympus) at 80%-90% confluency. For each well, 288 ng of the plasmid containing the vector of interest, 72 ng of pMD2.G and 144 ng of psPAX2 (gifts from Dr. Isaac Hilton's Lab at Rice University) were transfected using 1.0 μl of Lipofectamine 2000 and 25 μl Opti-MEM I reduced serum medium (Life Technologies). Viral supernatant was harvested 48 h post-transfection, filtered with a 0.45-μm PVDF filter (Millipore), then serially diluted to add into a 24-well plate cultured with 5×10⁴ HEK293T cells per well. After 24 h, cells transduced with lentivirus were split into new plate wells supplemented with 3 μg·ml⁻¹ puromycin. 72 h after the puromycin selection, cells were harvested from the well with the fewest surviving colonies to ensure single-copy integration and were then further cultured for expansion.

High-throughput sequencing (HTS) library preparation. The HTS library was prepared using two rounds of PCR. For the first round, a 200-bp DNA fragment of the target region was amplified in a total volume of 25 μL mixed with 12.5 μL of the Q5 High-Fidelity 2× Master Mix, 1 μL of the extracted genomic DNA, and a pair of primers (see Supplementary Materials). Successful amplification of individual samples was checked using 1% agarose gel. For the second round, combinations of different Illumina indexes were attached at each 5′ and 3′ end of the first PCR products using the same total PCR volume. The PCR products were combined and column-purified using a QIAquick PCR Purification Kit (Qiagen) and further gel extracted to remove non-specific amplifications. The final mixture of the library was quantified using Qubit dsDNA HS Assay Kit (Life Technologies) and prepared for loading into a 150-cycle MiSeq Reagent Kit v3 (Illumina) according to the manufacturer's protocol.

### Example 2 Enhancing A3G Activity

Previous studies show that the expression level of base editors in mammalian cells correlates with the enhanced base editing activity (Koblan et al., 2018). The wild-type human A3G (hA3G) DNA fragment was extracted from Addgene plasmid #113415 (hCMV_hA3G-BE3). The rAPOBEC1 deaminase portion of pCMV_BE4MAX (addgene #112093) was then replaced with the extracted A3G DNA fragment to construct hA3G(wild-type)-BE4MAX using the In-fusion method (Fisher #NC0465914). Additionally, the hA3G gene fragment was codon optimized and hA3G(opt.)-BE4MAX was constructed to even further improve the expression level.

Five mutations were incorporated into A3G (P200A+N236A+P247K+Q318K+Q322K) to improve the catalytic activity of A3G deaminase (Rathore et al., 2013; Maiti et al., 2008; Chen et al., 2008). These combined mutations in A3G improve the overall activity of C-to-T conversions without critically influencing the native ‘CC’ preferential motif. A gene fragment containing the five mutations was ordered from Genscript and cloned using the In-fusion method to construct A3G (opt.+P200A+N236A+P247K+Q318K+Q322K) BE4MAX. Additionally, the A3G deaminase was truncated to include only the C-terminal half (198-384) of the protein and all of the five mutations, which were in the C-terminal half. General PCR methods were used to clone the truncated A3G (198-384, opt.+P200A+N236A+P247K+Q318K+Q322K) BE4MAX.

### Example 3 Recovering ‘CC’ Selectivity

Activity enhancement generally results in relaxation of the ‘CC’ selectivity, meaning the C on the left, or the non-target C, is also likely to be converted to T. As such, the stringency of selectivity was increased by reducing the DNA binding energy around the target C region. Mutations of N244G and Y315F were individually introduced to the A3G constructs using PCR.

To test the C-to-T editing abilities of the engineered A3G base editors, various genomic sites that harbor the ‘CC’ dinucleotide sequence motif within the 5 nt activity window in HEK293T cells were selected. For these experiments, 35,000 HEK293T cells per well were cultured in 48-well plate (Corning #356509) under 250 μL of DMEM supplemented with 10% FBS (ThermoFisher #10437028) and 1× penicillin-streptomycin (ThermoFisher #15140122). The next day, HEK293T cells were transfected with 750 ng of base editor and 250 ng of target-specific guide RNA per well using lipofectamine 2000 (ThermoFisher #11668027). Cells were harvested after 3 days of transfection, genomic DNA extracted, and the region of the genomic site being tested amplified for Sanger sequencing in Genewiz. The chromatogram result from each Sanger sequence was inputted into EditR (Kluesner et al., 2018) to quantify the C-to-T conversions for both Cs on the left and right of the target sequence.

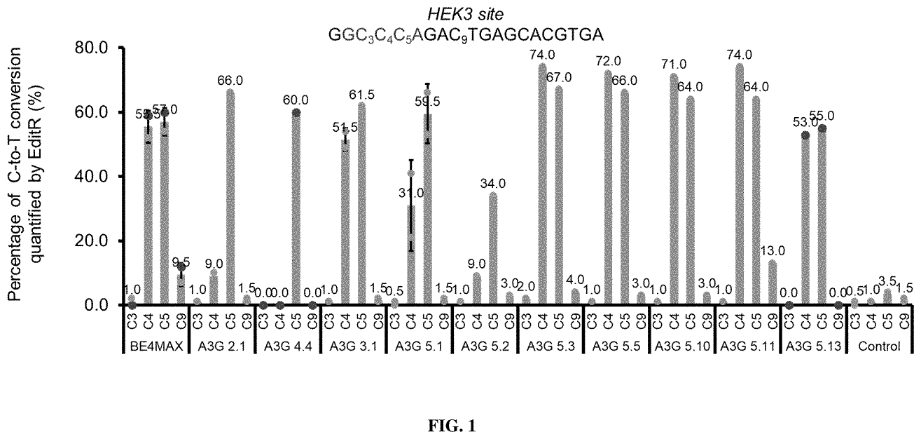

The HEK3 site (GGC₃C₄C₅AGC₉TGAGCACGTGA) was selected as a genomic site for testing. As shown in FIG. 1, A3G 2.1 and A3G 4.4, both of which contain no amino acid mutation in the A3G domain, displayed high selectivity for editing C5. All the other variants tested showed relaxed editing on both C4 and C5.

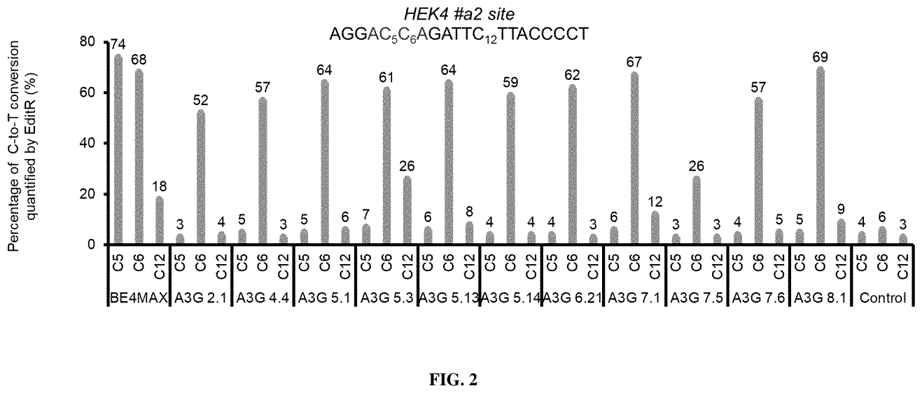

The ACCA motif in the HEK4 #a2 site (AGGAC₅C₆AGATTC₁₂TTACCCCT) was selected as a genomic site for testing. As shown in FIG. 2, C₆ was selectively edited over C₅ by all A3G variants tested. Note that CCA is a pseudo-native motif of A3G.

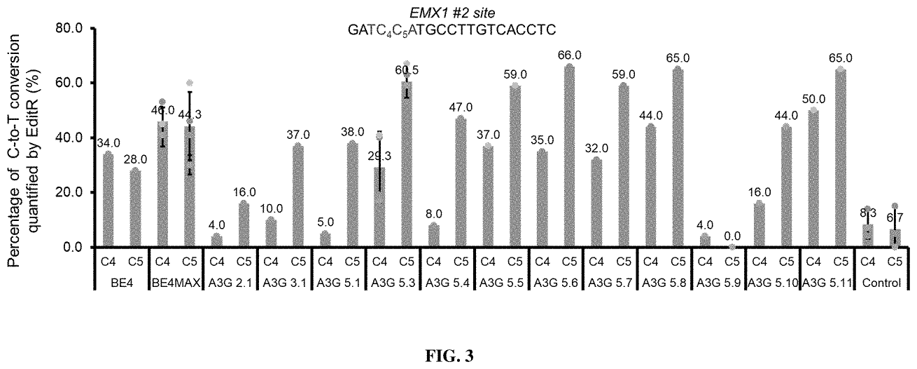

The TCCA motif in the EMX1 #2 site (GATC₄C₅ATGCCTTGTCACCTC) was selected as a genomic site for testing. As shown in FIG. 3, A3G 2.1, A3G 3.1, A3G 5.1, and A3G 5.4 showed selective editing of C₅ over C₄. All other variants displayed relaxation of selectivity, editing C₄ around or above half of the C₅ editing rate. This trend is a little different than that observed in the HEK4 #a2 site, which contained an ACCA motif. T at the −2 position from the target C in the TCCA motif in EMX1 #2 may contribute to this relaxation due to its similar structure as C.

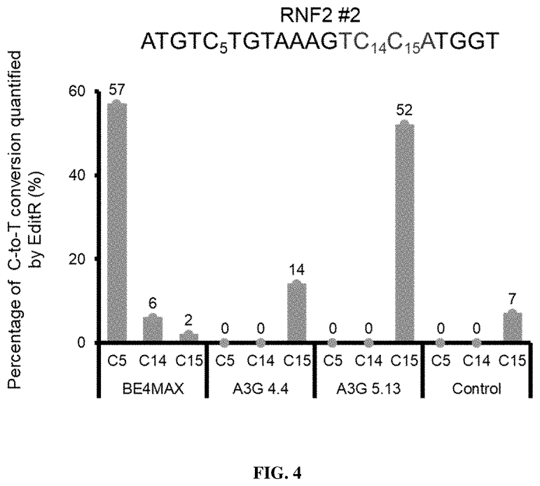

The TCCA motif in the RNF #2 site (ATGTC₅TGTAAAGTC₁₄C₁₅ATGGT) was selected as a genomic site for testing. This motif is the same as the previous EMX1 #2 site; however, this motif is outside the canonical window (positions from 4 to 9 while counting PAM as positions 21 and 23). As shown in FIG. 4, significant selectivity was observed for both A3G 4.4 and A3G 5.13, while high editing efficiency comparable to BE4MAX was observed for A3G 5.13. This suggests that A3G 5.13 might be more potent in editing single target C located outside the conventional activity window of BE4MAX.

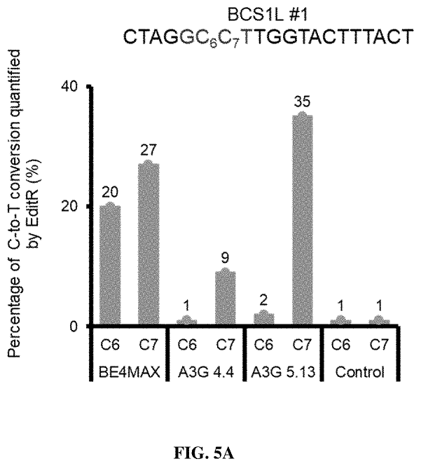

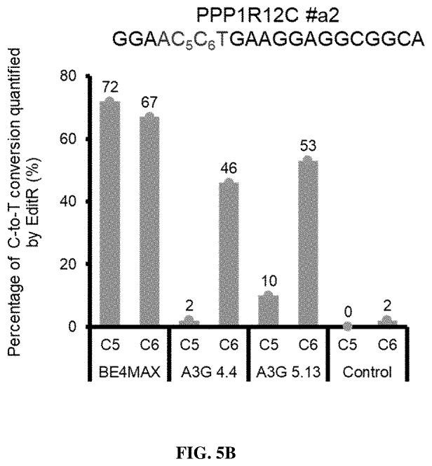

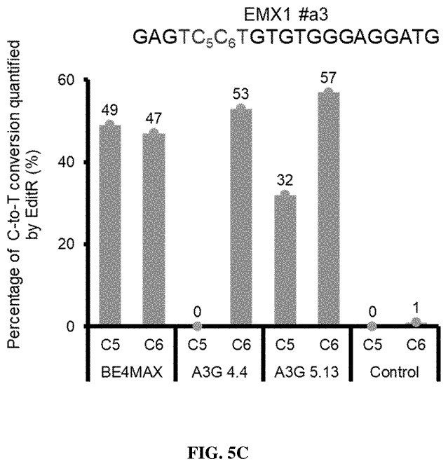

The GCCT motif in the BCS1L #1 site (CTAGGC₆C₇TTGGTACTTTACT), the ACCT motif in the PPP1R12C #a2 site (GGAAC₅C₆TGAAGGAGGCGGCA), and the TCCT motif in EMX1 #a3 site (GAGTC₅C₆TGTGTGGGAGGATG) were selected as genomic sites for testing. As shown in FIG. 5, A3G 4.4 displayed high selectivity between the two consecutive Cs across all three sites. In the BCS1L #1 site, which contains G at the −2 position from the target C, A3G 5.13 showed good editing efficiency and selectivity. A3G 5.13 selectively edited the second C over the first C in both BCS1L #1 and PPP1R12C #a2 sites, but not in EMX1 #a3, due to there being a T at the −2 position, which is structurally similar to C.

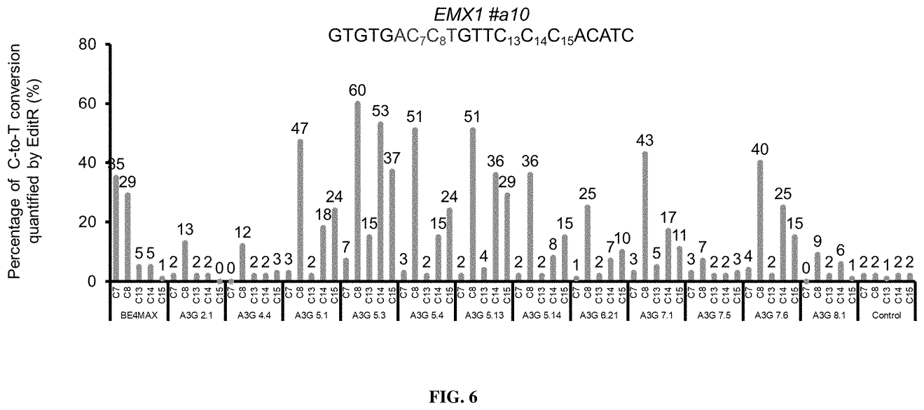

The ACCT motif in the EMX1 #a10 site (GTGTGAC₇C₈TGTTC₁₃C₁₄C₁₅ACATC) was selected as a genomic site for testing. As shown in FIG. 6, A3G 2.1 and 4.4 showed generally low activity. All other variants, except for A3G 7.5, displayed selective editing for C₈ over C₇. Variants 5.1, 5.3, 5.4, and 5.13 have greater activity than BE4MAX.

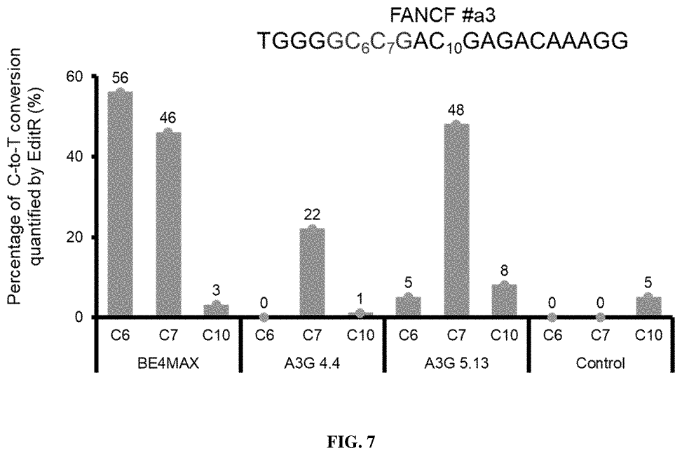

The GCCG motif in the FANCF #a3 site (TGGGGC₆C₇GAC₁₀GAGACAAAGG) was selected as a genomic site for testing. As shown in FIG. 7, A3G 5.13 selectively edited C₇ over C₆ with the editing efficiency of C₇ being comparable to that of BE4MAX. While A3G 4.4 showed high selectivity, the on-target editing rate was low. This trend is similar to that shown for the BCS1L #1 site that contained G at −2 position from the target C.

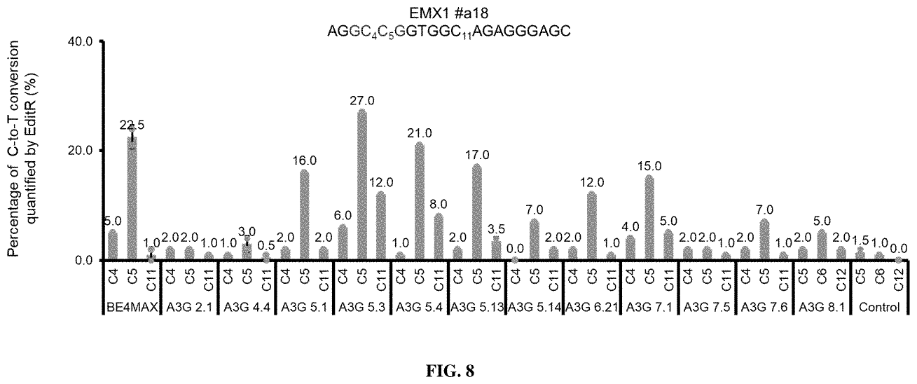

The GCCG motif in the EMX1 #a18 site (AGGC₄C₅GGTGGC₁₁AGAGGGAGC) was selected as a genomic site for testing. As shown in FIG. 8, both A3G 2.1 and A3G 4.4 shows low editing efficiency. However, A3G 5.1, A3G 5.4, and A3G 5.13 showed good selectivity while maintaining the on-target Cs editing above half of that of BE4MAX.

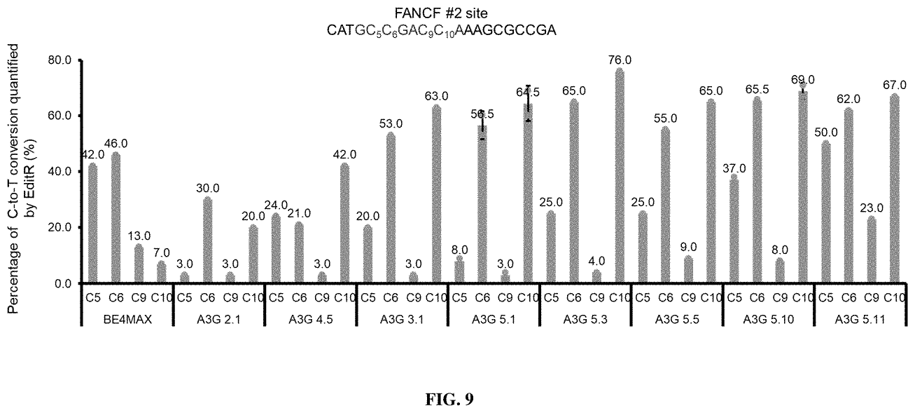

The GCCG and ACCA motifs in the FANCF #2 site (CATGC₅C₆GAC₉C₁₀AAAGCGCCGA) were selected as genomic sites for testing. As shown in FIG. 9, for all variants except for A3G 2.1, both the selectivity and editing efficiency were superior in the ‘ACCA’ than ‘GCG’ motif in FANCF #2 site.

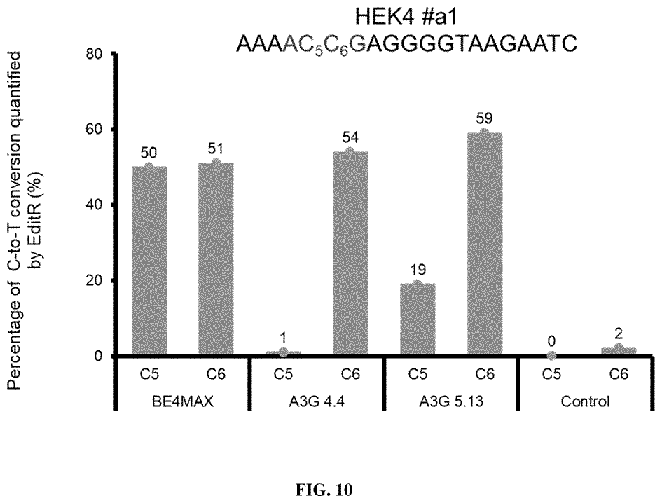

The ACCG motif in the HEK4 #a1 site (AAAAC₅C₆GAGGGGTAAGAATC) was selected as a genomic site for testing. As shown in FIG. 10, A3G 4.4 and 5.13 showed greater editing efficiency on C₆ than that of BE4MAX, while A3G 4.4 showed better selectivity on C₆ over C₅ than A3G 5.13.

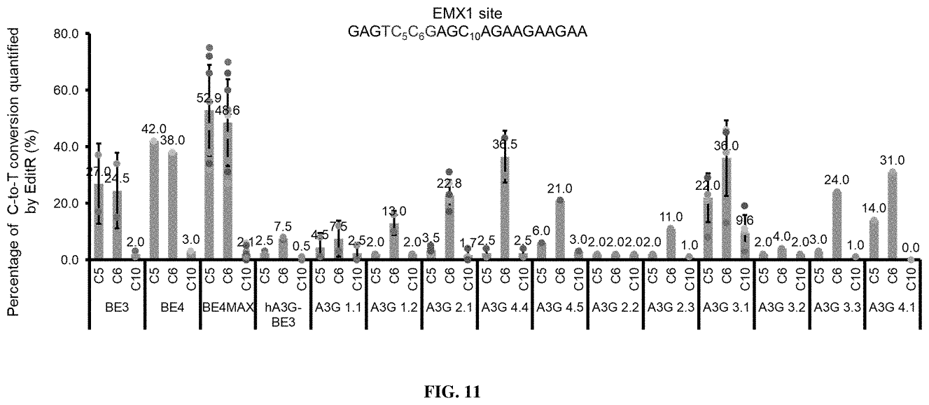

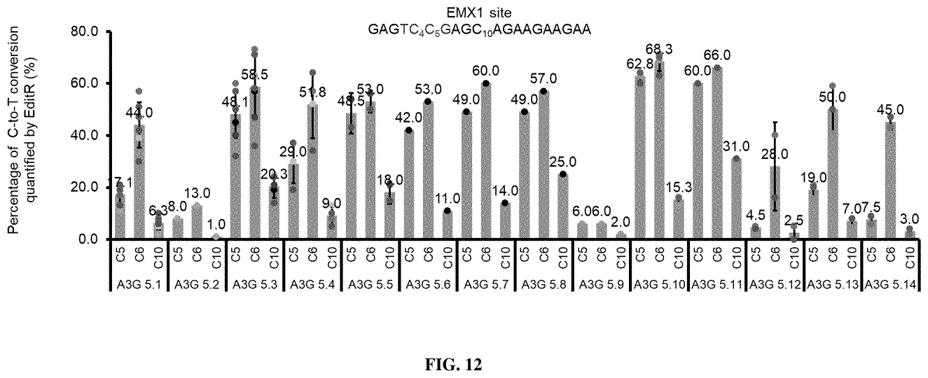

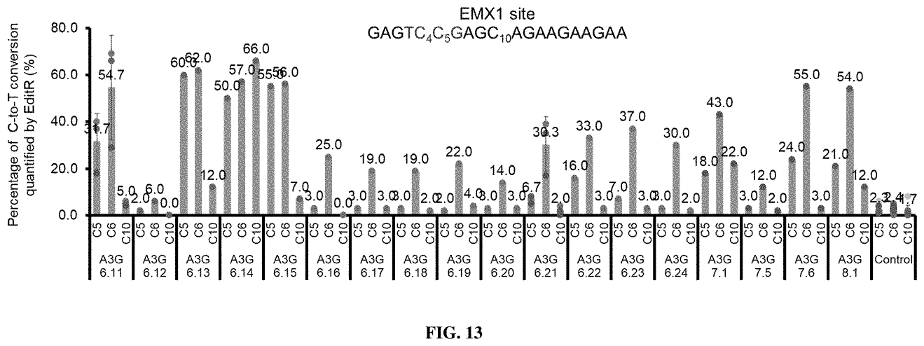

The TCCG motif in the EMX1 genomic site in HEK293T cells, which harbors the ‘CC’ dinucleotide sequence motif within the 5 nt activity window (EMX1: GAGTC₅C₆GAGC₁₀AGAAGAAGAA) was selected as one of the genomic sites for testing. As shown in FIG. 11, A3G 4.4 showed good selectivity on this site. Starting from A3G 5.1, a series of mutations were made to improve general catalytic efficiency of A3G base editor (Table 1). As shown in FIG. 12, A3G 5.10 and 5.11 displayed the highest editing activity. A3G 5.13 and 5.14, derived by adding Y315F mutation in A3G 5.3 and 5.4, respectively, displayed selectivity recovery while mildly compromising its general editing efficiency. As shown in FIG. 13, A3G version 6 base editor variants (6.11˜6.21) derived from A3G 5.10 showed that fine tuning could further enable selectivity enhancement by compromising its editing efficiency. A3G 8.1, which included the high-fidelity HypaCas9 variant from A3G 5.13, showed comparable editing rate on both C₄ and C₅ as that of A3G 5.13.

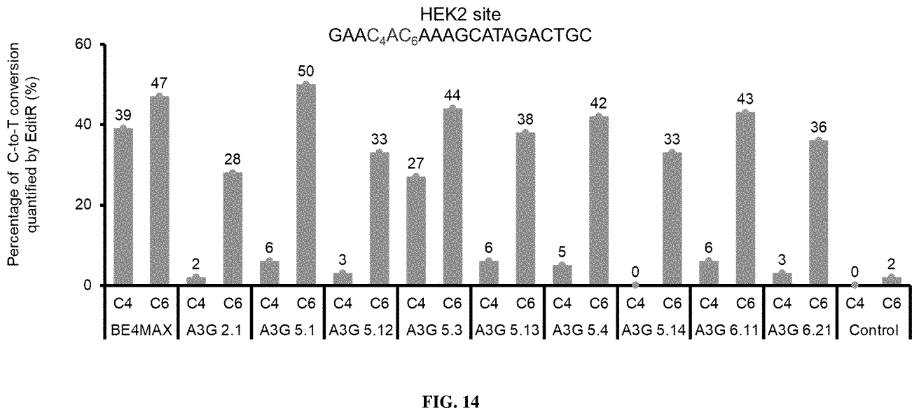

A3G natively prefers full motif of C₋₂C₋₁C₀A₊₁. Given that A3G prefers a C at −2 position, it was sought to determine whether the present variants can still selectively edit the target C₀ when the C₋₁ is changed to other nucleotides, including T, A, or G. To this end, the ACACA motif in the HEK2 site (GAAC₄AC₆AAAGCATAGACTGC) was selected as one of the genomic sites for testing. The HEK2 site represents a motif where two Cs are separated by one non-C nucleotide. As shown in FIG. 14, selective editing on C₆ over C₄ was observed from all variants except for A3G 5.3. As such, for sites harboring C₋₂ TC₀, C₋₂ AC₀, and C₋₂ GC₀, the present variants could preferably edit C₀ over C₋₂.

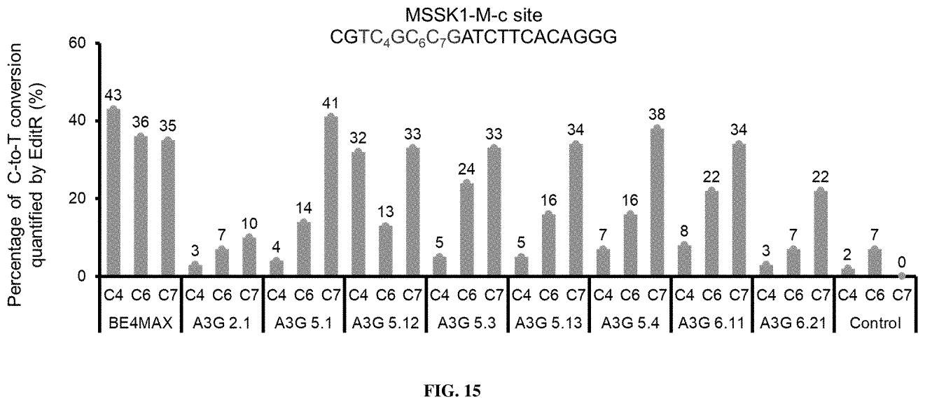

The TCGCCG motif in the MSSK1-M-c site (CGTC₄GC₆C₇GATCTTCACAGGG) was selected as one of the genomic sites for testing. In the MSSK1-M-c site, C₆ is more prone to be edited due to presence of C₄. As shown in FIG. 15, relaxed editing on C₆ was observed across all variants. However, editing rate on C₄ stayed low except for that edited by A3G 5.12, and BE4MAX displayed selective editing on all C₄, C₆, and C₇.

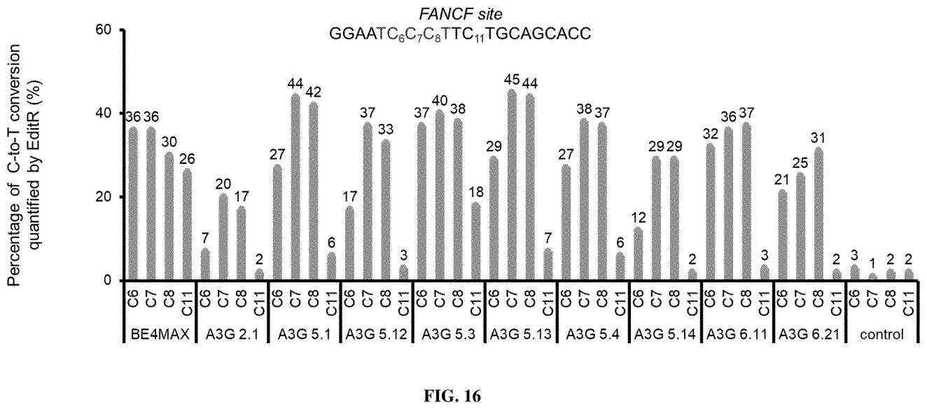

The TCCCT motif in the FANCF site (GGAATC₆C₇C₈TTC₁₁TGCAGCACC) was selected as one of the genomic sites for testing. As shown in FIG. 16, all variants including A3G 2.1 showed relaxed activity, although in general C₆ editing rates are lower than those of C₇ and C₈. Editing efficiencies between C₇ and C₈ are similar across all A3G variants. Importantly, T is structurally similar to C, indicating A3G could recognize it as close to its ideal motif. Ts in FANCF site might contribute to this trend of relaxation.

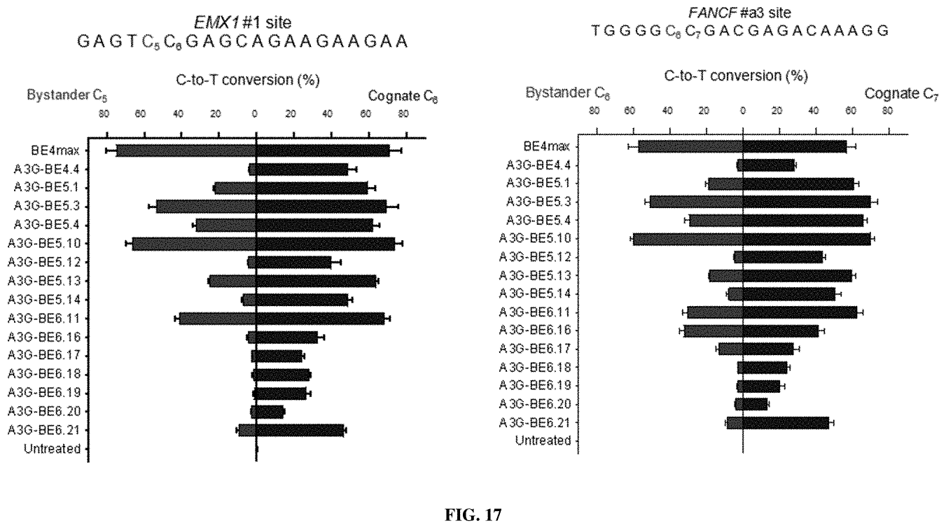

EMX1 and FANCF #a3 sites, which contain dinucleotide Cs (C5 and C6 of EMX1 #1 and C6 and C7 of FANCF #a3) within the conanical BE4max activity window, were selected to screen base editing efficiency and specificity of engineered A3G-BE variants in HEK293T cells. As shown in FIG. 17, A3G-BE6.11 induced higher selectivity than A3G-BE5.10 by moderately reducing editing of the bystander Cs, while A3G-BE6.16 and 6.17 displayed drastically reduced editing efficiencies of the cognate Cs, even below those of A3G-BE4.4. All A3G-BE6.18, 6.19, 6.20, and 6.21 showed improved editing ratios of the cognate to bystander Cs compared with A3G-BE6.11, their cognate C editing efficiencies did not outperform A3G-BE4.4. A3G-BE 5.13 and 5.14 cognate Cs editing efficiencies outperformed A3G-BE4.4, while bystander Cs editing efficiencies were appreciably low.

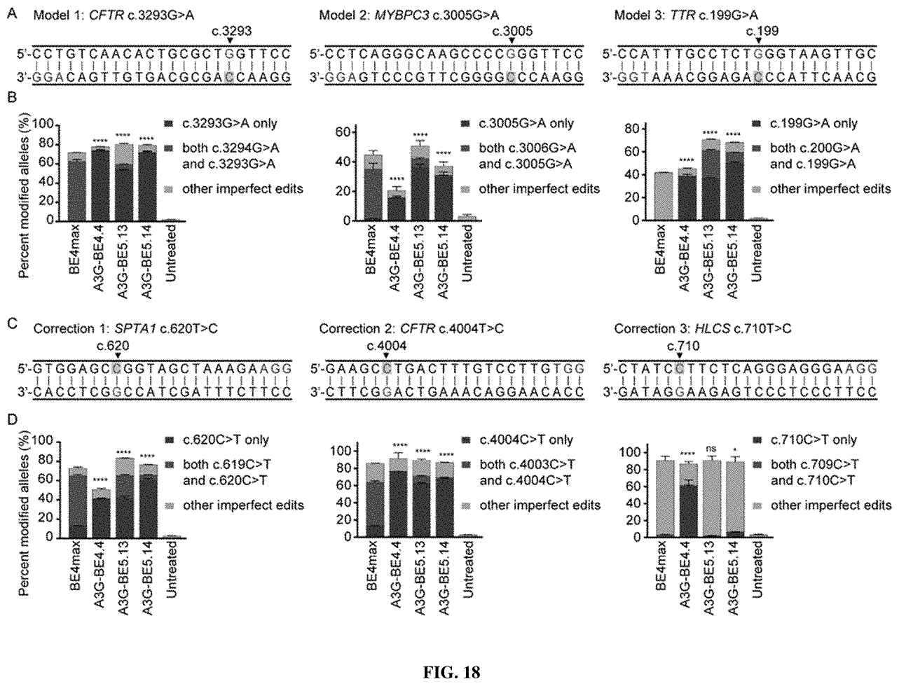

Three genetic variants caused by C-to-T (or G-to-A) substitution in which the wild-type sequences lie within the preferential 5′-CC-3′ motif of A3G-BEs were selected, including cystic fibrosis (model 1, NM_000492.3(CFTR):c.3293G>A), hypertonic myopathy (model 2, NM_000256.3(MYBPC3):c.3005G>A), and transthyretin amyloidosis (model 3, NM_000371.3(TTR):c.199G>A). Three reported human pathogenic SNPs caused by T>C (or A>G) mutations, which can be preferentially targeted by A3G-BEs were selected, including hereditary pyropoikilocytosis (correction 1, NM_003126.4(SPTA1):c.620T>C), cystic fibrosis (correction 2, NM_000492.3(CFTR):c.4004T>C), and holocarboxylase synthetase deficiency (correction 3, NM_000411.8(HLCS):c.710T>C). As shown in FIG. 18, HTS analysis was performed to quantify perfectly modified alleles for disease modeling and correction after treatment of BE4max, A3G-BE4.4, A3G-BE5.13, and A3G-BE5.14.

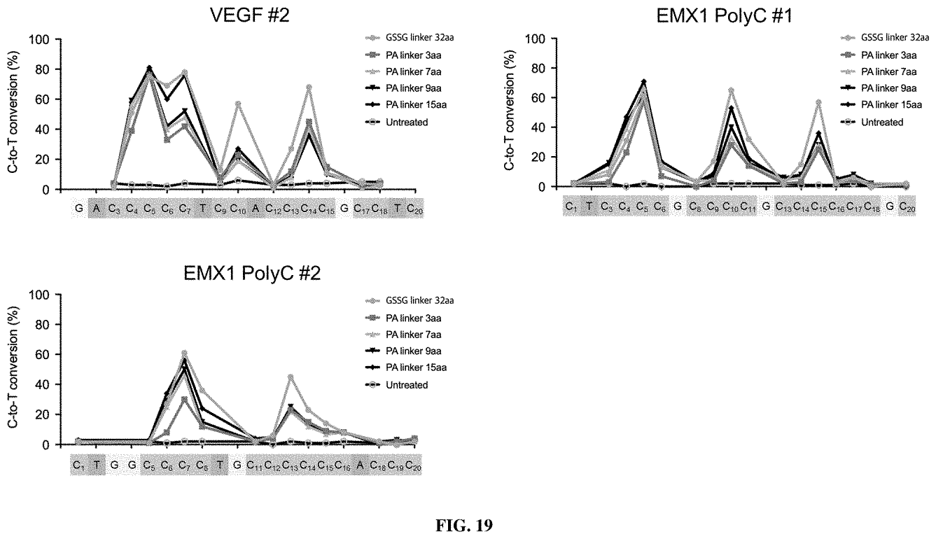

Although the engineered A3G BEs could target the second ‘C’ in the ‘CC’ motif that two consecutive ‘C’s exist, the activity windows are broad, e.g., expanding to 12 nucleotides. To further improve and narrow the activity window, starting from A3G-BE5.13, a series of engineered enzymes was generated to have a narrower window for precision-gene editing. To do so, A3G-BEs were engineered to incorporate more rigid linkers between the deaminase and nCas9, e.g., peptides with repetitive proline-alanine (PA) amino acid sequences (‘PA,’ or ‘PAPA,’ or ‘PAPAPA’), to provide conformational restriction. The repetitive ‘PA’ linkers (3, 7, 9 and 15 amino acids) were tested to directly compare the effects of both the length and rigidity of the linkers on the base editing outcomes. The activity window under the most stringent conditions was examined by testing endogenous EMX1 polyC #1 site that harbors multiple consecutive ‘C’s in HEK293T (FIG. 19). Base editing efficiencies were evaluated to correlate different linkers and their activity window sizes. When PA linker 3aa (PAP, named CBE-A3G5.20) was used, the editing efficiency at C₅ was comparable with the original GSSG linker 32aa, while bystander editing at other positions was significantly reduced.

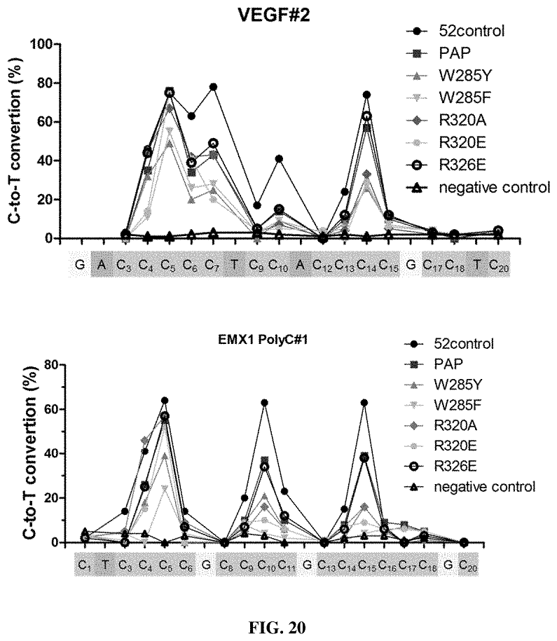

Next, different mutations were introduced to the CBE-A3G5.20 protein, including W285Y, W285F, R320A, R320E, and R326E. The new mutant with R320E mutation (named CBE-A3G5.28) maintained high editing activity at the C₅ position, while diminished the activity at other positions toward the 5′ end of the protospacer (FIG. 20).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

### REFERENCES

The following references, to the extent that they provide exemplary procedural or other details supplementary to those set forth herein, are specifically incorporated herein by reference.
U.S. Pat. No. 9,068,179
U.S. Pat. No. 9,840,699
U.S. Pat. No. 10,113,163
U.S. Pat. No. 10,167,457
U.S. Patent Application Publication 2018/0170984
U.S. Patent Application Publication 2018/0312828
PCT Patent Application Publication WO2018/165629
Anzalone et al., Search-and-replace genome editing without double-strand breaks or donor DNA. Nature, (2019).
Beale et al., Comparison of the differential context-dependence of DNA deamination by APOBEC enzymes: correlation with mutation spectra in vivo. Journal of molecular biology 337, 585-596 (2004).
Carpenter, M. A. et al. Methylcytosine and normal cytosine deamination by the foreign DNA restriction enzyme APOBEC3A. The Journal of Biological Chemistry 287, 34801-34808 (2012).
Chen, K.-M. et al. Structure of the DNA deaminase domain of the HIV-1 restriction factor APOBEC3G. Nature 452, 116-119 (2008).
Cheng et al., Expanding CT base editing toolkit with diversified cytidine deaminases. Nature communications 10, 3612 (2019).
Clement et al., CRISPResso2 provides accurate and rapid genome editing sequence analysis. Nature biotechnology 37, 224-226 (2019).
Conticello, The AID/APOBEC family of nucleic acid mutators. Genome biology 9, 229 (2008).
Gaudelli, N. M. et al. Programmable base editing of A T to G C in genomic DNA without DNA cleavage. Nature 551, 464-471 (2017).
Gehrke, J. M. et al. An APOBEC3A-Cas9 base editor with minimized bystander and off-target activities. Nature biotechnology 36, 977-982 (2018).
Grunewald, J. et al. Transcriptome-wide off-target RNA editing induced by CRISPR-guided DNA base editors. Nature 569, 433-437 (2019).
Harris et al., DNA deamination mediates innate immunity to retroviral infection. Cell 113, 803-809 (2003).
Holden et al., Crystal structure of the anti-viral APOBEC3G catalytic domain and functional implications. Nature 456, 121-124 (2008).
Huang, T. P. et al. Circularly permuted and PAM-modified Cas9 variants broaden the targeting scope of base editors. Nature Biotechnology (2019).
Hunt, in Single Nucleotide Polymorphisms. (Humana Press, Totowa, N.J., 2009), vol. 578, pp. 23-39.
Jiang, W. et al. BE-PLUS: a new base editing tool with broadened editing window and enhanced fidelity. Cell Research 28, 855-861 (2018).
Jin, S. et al. Cytosine, but not adenine, base editors induce genome-wide off-target mutations in rice. Science 364, 292-295 (2019).
Kim, Y. B. et al. Increasing the genome-targeting scope and precision of base editing with engineered Cas9-cytidine deaminase fusions. Nature biotechnology 35, 371-376 (2017).
Koblan, L. W. et al. Improving cytidine and adenine base editors by expression optimization and ancestral reconstruction. Nature Biotechnology 36, 843-846 (2018).
Komor, A. C., Badran, A. H. & Liu, D. R. CRISPR-based technologies for the manipulation of eukaryotic genomes. Cell 168, 20-36 (2017).
Komor, A. C. et al. Improved base excision repair inhibition and bacteriophage Mu Gam protein yields C: G-to-T: A base editors with higher efficiency and product purity. Science Advances 3, eaao4774 (2017).
Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
Landrum, M. J. et al. ClinVar: public archive of interpretations of clinically relevant variants. Nucleic Acids Research 44, D862-D868 (2016).
Langlois et al., Mutational comparison of the single-domained APOBEC3C and double-domained APOBEC3F/G anti-retroviral cytidine deaminases provides insight into their DNA target site specificities. Nucleic acids research 33, 1913-1923 (2005).
Lee, H. K. et al. Targeting fidelity of adenine and cytosine base editors in mouse embryos. Nature Communications 9, 4804 (2018).
Liu, Z., Chen, S., Shan, H., Chen, M., Song, Y., Lai, L., & Li, Z. (2019). Highly precise base editing with CC context-specificity using engineered human APOBEC3G-nCas9 fusions. BioRxiv. doi:10.1101/658351
Maiti, A. et al. Crystal structure of the catalytic domain of HIV-1 restriction factor APOBEC3G in complex with ssDNA. Nature Communications 9, 2460 (2018).
Marx, A., Galilee, M., & Alian, A. (2015). Zinc enhancement of cytidine deaminase activity highlights a potential allosteric role of loop-3 in regulating APOBEC3 enzymes. Scientific Reports, 5(1).
Morse et al., Dimerization regulates both deaminase-dependent and deaminase-independent HIV-1 restriction by APOBEC3G. Nature communications 8, 597 (2017).
Nishida, K. et al. Targeted nucleotide editing using hybrid prokaryotic and vertebrate adaptive immune systems. Science 353, aaf8729 (2016).
Nowarski et al., APOBEC3G inhibits HIV-1 RNA elongation by inactivating the viral trans-activation response element. Journal of molecular biology 426, 2840-2853 (2014).
Ran et al., Genome engineering using the CRISPR-Cas9 system. Nature protocols 8, 2281-2308 (2013).
Rathore, A. et al. The local dinucleotide preference of APOBEC3G can be altered from 5′-CC to 5′-TC by a single amino acid substitution. Journal of Molecular Biology 425, 10.1016/j.jmb.2013.1007.1040 (2013).
Rees, H. A. et al. Improving the DNA specificity and applicability of base editing through protein engineering and protein delivery. Nature Communications 8, 15790 (2017).
Rees, H. A. & Liu, D. R. Base editing: precision chemistry on the genome and transcriptome of living cells. Nature Reviews Genetics 19, 770-788 (2018).
Ryu et al., Adenine base editing in mouse embryos and an adult mouse model of Duchenne muscular dystrophy. Nature biotechnology 36, 536 (2018).
Tan et al., Engineering of high-precision base editors for site-456 specific single nucleotide replacement. Nature communications 10, 439 (2019).
Tanenbaum et al., A protein-tagging system for signal amplification in gene expression and fluorescence imaging. Cell 159, 635-646 (2014).
Wang, X. et al. Efficient base editing in methylated regions with a human APOBEC3A-Cas9 fusion. Nature Biotechnology 36, 946-949 (2018).
Wang et al., Comparison of cytosine base editors and development of the BEable-GPS database for targeting pathogenic SNVs. Genome biology 20, 1-7 (2019).
Yu et al., Single-strand specificity of APOBEC3G accounts for minus-strand deamination of the HIV genome. Nature structural & molecular biology 11, 435-442 (2004).
Zhou et al., Off-target RNA mutation induced by DNA base editing and its elimination by mutagenesis. Nature 571, 275-278 (2019).
Ziegler et al., Insights into DNA substrate selection by APOBEC3G from structural, biochemical, and functional studies. PLoS One 13, e0195048 (2018).
Zong, Y. et al. Efficient C-to-T base editing in plants using a fusion of nCas9 and human APOBEC3A. Nature biotechnology 36, 950-953 (2018).
Zuo, E. et al. Cytosine base editor generates substantial off-target single-nucleotide variants in mouse embryos. Science 364, 289-292 (2019).

1. A polypeptide comprising a variant of a native cytosine deaminase (CD) domain, wherein the variant CD domain comprises a sequence at least 90% identical to amino acids 198-384 of SEQ ID NO: 48, and comprises P200A, N236A, P247K, Q318K, Q322K substitutions relative to SEQ ID NO: 48.

2. The polypeptide of claim 1, further comprising a Y315F or a N244G substitution relative to SEQ ID NO: 48.

3. The polypeptide of claim 1, further comprising H248N, K249L, H250L, G251C, F252G, L253F, and E254Y substitutions relative to SEQ ID NO: 48.

4-9. (canceled)

10. The polypeptide of claim 1, further comprising L234K, F310K, C243A, C321A, and C356A substitutions relative to SEQ ID NO: 48.

11-13. (canceled)

14. The polypeptide of claim 1, wherein the CD domain is a chimpanzee, gorilla, monkey, cow, dog, rat, mouse, or human APOBEC3G deaminase domain.

15. The polypeptide of claim 1, wherein the polypeptide further comprises a Cas9 domain that has nickase activity.

16-20. (canceled)

21. The polypeptide of claim 15, wherein the polypeptide further comprises a uracil glycosylase inhibitor (UGI) domain.

22-23. (canceled)

24. The polypeptide of claim 1, wherein the polypeptide further comprises a nuclear localization sequence.

25. (canceled)

26. A nucleic acid comprising a nucleotide sequence encoding the polypeptide of claim 1.

27-32. (canceled)

33. A host cell comprising the nucleic acid of claim 26.

34-36. (canceled)

37. A viral vector comprising the nucleic acid of claim 26.

38-43. (canceled)

44. A composition comprising the nucleic acid of claim 26 and a nucleic acid encoding a guide RNA.

45-53. (canceled)

54. A composition comprising the polypeptide of claim 15 and a guide RNA bound to the Cas9 domain of the polypeptide.

55-57. (canceled)

58. A method for targeted modification of a selected DNA sequence, the method comprising contacting the DNA sequence with a polypeptide of claim 15 and a nucleic acid comprising a guide RNA (gRNA) sequence targeted to the selected DNA sequence, where the gRNA complexed with the polypeptide and directs the polypeptide to the selected DNA sequence, wherein the targeted modification is the deamination of a deoxycytidine within the selected DNA sequence.

59. (canceled)

60. The method of claim 58, wherein the selected sequence comprises a CC motif.

61. The method of claim 60, wherein the targeted modification is the deamination of the second deoxycytidine within the CC motif.

62. The method of claim 58, wherein the selected sequence comprises a DCYD motif or a DCCYD motif, wherein D represent A, G, or T, and wherein Y denotes the T>C mutation.

63. (canceled)

64. The method of claim 58, wherein the selected sequence comprises a CCCA motif, wherein the targeted modification is the deamination of the third deoxycytidine within the motif.

65-66. (canceled)

67. The method of claim 58, wherein the contacting is in vivo in a subject identified as having a clinical condition, wherein the selected DNA sequence is associated with the clinical condition, and wherein the deamination corrects a point mutation in the selected DNA sequence associated with the clinical condition.

68. (canceled)

69. The method of claim 67, wherein the clinical condition is hereditary pyropoikilocytosis, cystic fibrosis, or holocarboxylase synthetase deficiency.

70-72. (canceled)
